SEQUENCE LISTING (81 sequences)

SEQ ID NO: 1 - protein, 533 aa, Homo sapiens
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 2 - protein, 609 aa, Homo sapiens [sequence omitted]

SEQ ID NO: 3 - protein, 8 aa, synthetic construct
GRTFIAYA

SEQ ID NO: 4 - protein, 9 aa, synthetic construct
ITNFAGGTT

SEQ ID NO: 5 - protein, 18 aa, synthetic construct
AADRSAQTMRQVRPVLPY

SEQ ID NO: 6 - protein, 126 aa, synthetic construct [sequence omitted]

SEQ ID NO: 7 - protein, 126 aa, synthetic construct [sequence omitted]

SEQ ID NO: 8 - protein, 25 aa, synthetic construct
  VARIANT 1..25: note = This sequence may encompass 3-5 GGGGS repeating units
GGGGSGGGGSGGGGSGGGGSGGGGS

SEQ ID NO: 9 - protein, 15 aa, synthetic construct
GGGGSGGGGSGGGGS

SEQ ID NO: 10 - protein, 648 aa, synthetic construct
  VARIANT 127..151: note = This region may encompass 3-5 GGGGS repeating units
QVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSGGGGSGGGGSSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 11 - protein, 648 aa, synthetic construct
  VARIANT 127..151: note = This region may encompass 3-5 GGGGS repeating units
QVQLVESGGGLVQPGGSLRLSCAASGRTFIAYAMGWFRQAPGKEREFVAAITNFAGGTTYYADSVKGRFTISRDNAKTTVYLQMNSLRAEDTAVYYCAADRSAQTMRQVRPVLPYWGQGTLVTVSSGGGGSGGGGSGGGGSGGGGSGGGGSSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 12 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 13 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 232 bp, synthetic construct
  source 1..232: mol_type = other DNA
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagg

SEQ ID NO: 15 - DNA, 522 bp, synthetic construct
  source 1..522: mol_type = other DNA
tcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 16 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 17 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 18 - protein, 657 aa, synthetic construct
MELGLSWVVLAALLQGVQAQVQLVESGGGLVQAGGSLRLSCVASGRTFIAYAMGWFRQAPGKEREFVAAITNFAGGTTYYADSVKGRFTISRDNAKTTVYLQMNSLKPEDTALYYCAADRSAQTMRQVRPVLPYWGQGTQVTVSSGGGGSGGGGSGGGGSSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 19 - DNA, 1974 bp, synthetic construct
  source 1..1974: mol_type = other DNA
atggagctggggctgagctgggtggtcctggctgctcttttacaaggtgtccaggctcaggtgcagctggtcgagagcgggggcgggctggtgcaggccggaggaagcctgagactgtcttgcgtggccagcggcagaacctttatcgcttacgccatgggctggtttagacaggccccaggaaaagagcgggagtttgtggcagccatcacaaatttcgccggcggtaccacctactacgccgacagcgtgaagggcagattcaccatttctagagacaacgccaagacaacagtgtacctgcagatgaattctctgaaacctgaggacacagccctgtattattgcgccgccgaccgcagtgcccagactatgaggcaggtgaggcctgtgctgccctattggggacagggcactcaggtgaccgtgagcagcggtggtggcggttcaggcggaggtggctctggcggtggcggatcgtcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtag

SEQ ID NO: 20 - DNA, 6716 bp, synthetic construct
  source 1..6716: mol_type = other DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggagctggggctgagctgggtggtcctggctgctcttttacaaggtgtccaggctcaggtgcagctggtcgagagcgggggcgggctggtgcaggccggaggaagcctgagactgtcttgcgtggccagcggcagaacctttatcgcttacgccatgggctggtttagacaggccccaggaaaagagcgggagtttgtggcagccatcacaaatttcgccggcggtaccacctactacgccgacagcgtgaagggcagattcaccatttctagagacaacgccaagacaacagtgtacctgcagatgaattctctgaaacctgaggacacagccctgtattattgcgccgccgaccgcagtgcccagactatgaggcaggtgaggcctgtgctgccctattggggacagggcactcaggtgaccgtgagcagcggtggtggcggttcaggcggaggtggctctggcggtggcggatcgtcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtagacccagctttcttgtacaaagtggtgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtggctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa

SEQ ID NO: 21 - protein, 46 aa, synthetic construct
LAEAKVLANRELDKYGVSDYYKNLINNAKTVEGVKALIDEILAALP

SEQ ID NO: 22 - protein, 9 aa, synthetic construct
GFLLRSNTM

SEQ ID NO: 23 - protein, 7 aa, synthetic construct
IRPSGLT

SEQ ID NO: 24 - protein, 10 aa, synthetic construct
HTRPPFQRDS

SEQ ID NO: 25 - protein, 10 aa, synthetic construct
ATRPPFQRDS

SEQ ID NO: 26 - protein, 9 aa, synthetic construct
GRTFDNYVM

SEQ ID NO: 27 - protein, 8 aa, synthetic construct
ISGSGSIT

SEQ ID NO: 28 - protein, 17 aa, synthetic construct
AAGSRRTYYREPKFYPS

SEQ ID NO: 29 - protein, 9 aa, synthetic construct
GSTFSSSSV

SEQ ID NO: 30 - protein, 7 aa, synthetic construct
ITSGGST

SEQ ID NO: 31 - protein, 18 aa, synthetic construct
NVAGRNWVPISRYSPGPY

SEQ ID NO: 32 - protein, 18 aa, synthetic construct
AVAGRNWVPISRYSPGPY

SEQ ID NO: 33 - protein, 116 aa, synthetic construct [sequence omitted]

SEQ ID NO: 34 - protein, 116 aa, synthetic construct
QVQLVESGGGLVQPGGSLRLSCAGPGFLLRSNTMGWYRQAPGKERELVAFIRPSGLTNYNDAVQGRFTISRDNAKNTVYLQMNSLRAEDTAVYYCHTRPPFQRDSWGQGTLVTVSS

SEQ ID NO: 35 - protein, 124 aa, synthetic construct [sequence omitted]

SEQ ID NO: 36 - protein, 124 aa, synthetic construct
EVQLVESGGGLVQPGGSLRLSCAASGRTFDNYVMAWFRQAPGKGLEFVSSISGSGSITNYANSVKDRFTISRDNSKNTLYLQMNSLRAEDTAVYYCAAGSRRTYYREPKFYPSWGQGTLVTVSS

SEQ ID NO: 37 - protein, 124 aa, synthetic construct [sequence omitted]

SEQ ID NO: 38 - protein, 124 aa, synthetic construct
EVQLVESGGGLVQPGGSLRLSCAASGSTFSSSSVGWYRQAPGKGLELVSAITSGGSTNTADSVKGRFTISRDNSKNTLYLQMNSLRAEDTAVYYCNVAGRNWVPISRYSPGPYWGQGTLVTVSS

SEQ ID NO: 39 - protein, 4 aa, synthetic construct
GGGG

SEQ ID NO: 40 - protein, 5 aa, synthetic construct
TGGGG

SEQ ID NO: 41 - protein, 20 aa, synthetic construct
GGSSGGSGSSSGSGGSGSSG

SEQ ID NO: 42 - protein, 28 aa, synthetic construct
  VARIANT 1..28: note = This sequence may encompass 1-7 GGSS repeating units
GGSSGGSSGGSSGGSSGGSSGGSSGGSS

SEQ ID NO: 43 - protein, 30 aa, synthetic construct
  VARIANT 1..30: note = This sequence may encompass 1-6 SGGGG repeating units
SGGGGSGGGGSGGGGSGGGGSGGGGSGGGG

SEQ ID NO: 44 - protein, 44 aa, synthetic construct
  VARIANT 5..44: note = This region may encompass 1-8 SGGGG repeating units
GGGGSGGGGSGGGGSGGGGSGGGGSGGGGSGGGGSGGGGSGGGG

SEQ ID NO: 45 - protein, 22 aa, synthetic construct
SKPTCPPPELLGGPSVFIFPPK

SEQ ID NO: 46 - protein, 16 aa, synthetic construct
GGGSGGGSGGGSGGGS

SEQ ID NO: 47 - protein, 20 aa, synthetic construct
GGGSGGGSGGGSGGGSGGGS

SEQ ID NO: 48 - protein, 19 aa, synthetic construct
MELGLSWVVLAALLQGVQA

SEQ ID NO: 49 - protein, 18 aa, Homo sapiens
MKWVTFISLLFLFSSAYS

SEQ ID NO: 50 - protein, 20 aa, Homo sapiens
MVLQTQVFISLLLWISGAYG

SEQ ID NO: 51 - protein, 23 aa, Homo sapiens
MQMSPALTCLVLGLALVFGEGSA

SEQ ID NO: 52 - protein, 18 aa, Homo sapiens
MGLAWGLGVLFLMHVCGT

SEQ ID NO: 53 - protein, 31 aa, Homo sapiens
MLRGPGPGLLLLAVQCLGTAVPSTGASKSKR

SEQ ID NO: 54 - protein, 21 aa, Homo sapiens
MGWRAAGALLLALLLHGRLLA

SEQ ID NO: 55 - protein, 29 aa, Homo sapiens
MAGRSHPGPLRPLLPLLVVAACVLPGAGG

SEQ ID NO: 56 - skipped

SEQ ID NO: 57 - protein, 5 aa, synthetic construct
RGDSP

SEQ ID NO: 58 - protein, 4 aa, synthetic construct
GRGD

SEQ ID NO: 59 - protein, 6 aa, synthetic construct
GRGDSP

SEQ ID NO: 60 - protein, 7 aa, synthetic construct
GGRGDSP

SEQ ID NO: 61 - protein, 8 aa, synthetic construct
GGGRGDSP

SEQ ID NO: 62 - protein, 29 aa, synthetic construct
  VARIANT 5..29: note = This region may encompass 1-5 SGGGG repeating units
GGGGSGGGGSGGGGSGGGGSGGGGSGGGG

SEQ ID NO: 63 - protein, 116 aa, synthetic construct
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 64 - protein, 115 aa, synthetic construct [sequence omitted]

SEQ ID NO: 65 - protein, 116 aa, synthetic construct
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 66 - protein, 114 aa, synthetic construct
AVQLVESGGGLVQPGGSLRLTCTASGFTFRSFGMSWVRQAPGKDQEWVSAISADSSTKNYADSVKGRFTISRDNAKKMLYLEMNSLKPEDTAVYYCVIGRGSPSSPGTQVTVSS

SEQ ID NO: 67 - protein, 114 aa, synthetic construct
QVQLAESGGGLVQPGGSLRLTCTASGFTFGSFGMSWVRQAPGEGLEWVSAISADSSDKRYADSVKGRFTISRDNAKKMLYLEMNSLKSEDTAVYYCVIGRGSPASQGTQVTVSS

SEQ ID NO: 68 - protein, 123 aa, synthetic construct
QVQLVESGGGLVQPGGSLRLSCAASGFTFSNYWMYWVRVAPGKGLERISRDISTGGGYSYYADSVKGRFTISRDNAKNTLYLQMNSLKPEDTALYYCAKDREAQVDTLDFDYRGQGTQVTVSS

SEQ ID NO: 69 - protein, 115 aa, synthetic construct [sequence omitted]

SEQ ID NO: 70 - protein, 115 aa, synthetic construct [sequence omitted]

SEQ ID NO: 71 - protein, 115 aa, synthetic construct [sequence omitted]

SEQ ID NO: 72 - protein, 115 aa, synthetic construct [sequence omitted]

SEQ ID NO: 73 - protein, 115 aa, synthetic construct [sequence omitted]

SEQ ID NO: 74 - protein, 115 aa, synthetic construct [sequence omitted]

SEQ ID NO: 75 - protein, 115 aa, synthetic construct [sequence omitted]

SEQ ID NO: 76 - protein, 115 aa, synthetic construct [sequence omitted]

SEQ ID NO: 77 - protein, 30 aa, synthetic construct
  VARIANT 1..30: note = This sequence may encompass 1-6 GGGGS repeating units
GGGGSGGGGSGGGGSGGGGSGGGGSGGGGS

SEQ ID NO: 78 - protein, 12 aa, synthetic construct
GGGSGGGSGGGS

SEQ ID NO: 79 - protein, 117 aa, synthetic construct
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 80 - protein, 32 aa, synthetic construct
  VARIANT 1..32: note = This sequence may encompass 1-8 GGSS repeating units
GGSSGGSSGGSSGGSSGGSSGGSSGGSSGGSS

SEQ ID NO: 81 - protein, 40 aa, synthetic construct
  VARIANT 1..40: note = This sequence may encompass 1-8 SGGGG repeating units
SGGGGSGGGGSGGGGSGGGGSGGGGSGGGGSGGGGSGGGG

(19) United States
(12) Patent Application Publication
(10) Pub. No.: US 2025/0136671 A1
(43) Pub. Date: May 1, 2025

(54) THERAPIES FOR MUCOPOLYSACCHARIDOSIS TYPE 6

(71) Applicants: SIGILON THERAPEUTICS, INC., Cambridge, MA (US); National Research Council of Canada, Ottawa (CA)

(72) Inventors: Elina Makino, Winchester, MA (US); Erika Pearson, Mansfield, MA (US); Gregory Hussack, Ottawa (CA)

(21) Appl. No.: 18/833,632

(22) PCT Filed: Jan. 27, 2023
(86) PCT No.: PCT/US2023/011738

§ 371 Date: Jul. 26, 2024

Related U.S. Application Data
(60) Provisional application No. 63/304,382, filed on Jan. 28, 2022.

(51) Int. Cl. C07K 16/18; A61K 9/48; A61K 35/12; A61K 38/46; A61K 47/36; A61P 3/00; C12N 9/16; C12N 15/62

(52) U.S. Cl. CPC C07K 16/18; A61K 9/48; A61K 35/12; A61K 38/465; A61K 47/36; A61P 3/00; C12N 9/16; C12N 15/62; C12Y 301/06012; C07K 2317/24; C07K 2317/92; C12N 2510/00

(57) ABSTRACT

Described herein are ARSB fusion proteins, polynucleotides encoding the fusion proteins and mammalian cells genetically modified to express and secrete the fusion proteins, as well as compositions, implantable devices and device preparations comprising the fusion proteins or genetically modified cells secreting the fusion proteins, and methods of making and using the same for treating MPS-6.

### CLAIM OF PRIORITY

The present application claims priority to U.S. Patent Application No. 63/304,382, filed on Jan. 28, 2022. The entire contents of the foregoing application is incorporated herein by reference in its entirety.

### BACKGROUND

Mucopolysaccharidosis type 6 (MPS-6) is a rare, autosomal genetic disease characterized by deficient activity of the lysosomal enzyme arylsulfatase B (ARSB) (also known as N-acetylglucosamine 4-sulfatase), which results in lysosome accumulation of the glycosaminoglycans (GAGs) dermatan sulfate (DS) and chondroitin sulfate (CS). Lysosome storage of DS and CS cause a number of problems including bone dysplasia, joint restriction, organomegaly, heart disease, and corneal clouding. MPS-6 typically presents in one of two forms: a rapidly advancing form, which leads to severe disease in the majority of patients, and a slowly-progressing form with more attenuated symptoms in a minority of patients.

The most widely used specific treatment for MPS-6 disease is enzyme replacement therapy (ERT), which currently is a life-long therapy that requires weekly or twice weekly intravenous infusions with a recombinant version of ARSB. Thus, novel treatment modalities for MPS-6 are desirable.

### SUMMARY

Described herein are ARSB fusion proteins that comprise a domain that binds to human serum albumin (HSA) and an amino acid sequence for a mammalian ARSB protein, e.g., the amino acid sequence for human mature ARSB. The HSA-binding domain is located N-terminal to the ARSB amino acid sequence.

In one aspect, the present disclosure features an ARSB fusion protein which comprises a primary structure defined by formula F: AB-L-ARSB, wherein AB is an HSA-binding domain, L is a linker moiety and ARSB comprises an amino acid sequence of a mature mammalian ARSB protein, e.g., amino acid 37 or 39 to amino acid 533 of FIG. 1 (SEQ ID NO: 1). In an embodiment, AB has a molecular weight of less than about any of 75 kDa, 50 kDa or 25 kDa. In an embodiment, AB comprises, consists essentially of, or consists of an amino acid sequence from a single chain Fab (scFab), a single chain Fv (scFv) or a single domain antibody (sdAb). In an embodiment, the linker moiety is a linker peptide that is less than 50 amino acids in length.

In an embodiment, the ARSB fusion protein binds via AB to domain 1 (DI) or domain 2 (DII) of HSA and does not substantially inhibit binding of human FcRn (h-FcRn) to HSA. In an embodiment, the fusion protein binds via AB to HSA with a K_{D} affinity of less than about 1 nM to about 100 nM within a pH range of about 5.0 to about 7.4 as determined by surface plasmon resonance at 25° C. In some embodiments, the fusion protein also binds via AB to at least one non-human mammalian serum albumin ortholog at 25° C. within a pH range of about 5.5 to about 7.4.

In an embodiment, the albumin ortholog is from mouse, rat, hamster, rabbit, guinea pig, pig, cat, dog, or a non-human primate (e.g., cynomolgus or rhesus monkey).

In an embodiment, AB comprises first, second and third amino acid sequences corresponding to the three complementarity determining regions CDR1, CDR2 and CDR3 of the heavy chain variable region of an anti-HSA antibody (e.g., a conventional antibody with two heavy chains and two light chains, an scFab, an scFv, a sdAb). In an embodiment, the CDR1, CDR2 and CDR3 amino acid sequences in AB are: GRTFIAYA (SEQ ID NO:3) or a conservatively substituted variant thereof, ITNFAGGTT (SEQ ID NO:4) or a conservatively substituted variant thereof, and AADRSAQTMRQVRPVLPY (SEQ ID NO:5) or a conservatively substituted variant thereof.

In an embodiment, AB consists essentially of, or consists of: QVQLVESGGGLVQAGGSLRLSCVASGRTFIAYAMGWFRQAPGKEREFVAAITNFAGGT TYYADSVKGRFTISRDNAKTTVYLQMNSLKPEDTALYYCAADRSAQTMRQVRPVLPY WGQGTQVTVSS (SEQ ID NO:6), or a conservatively substituted variant thereof. In an embodiment, AB consists essentially of, or consists of: QVQLVESGGGLVQPGGSLRLSCAASGRTFIAYAMGWFRQAPGKEREFVAAITNFAGGT TYYADSVKGRFTISRDNAKTTVYLQMNSLRAEDTAVYYCAADRSAQTMRQVRPVLPY WGQGTLVTVSS (SEQ ID NO:7), or a conservatively substituted variant thereof. In an embodiment, AB consists essentially of, or consists of, an amino acid sequence of the heavy chain variable region of an antibody that cross-competes with a sdAb consisting of SEQ ID NO:6 or SEQ ID NO:7 for binding to HSA.

The linker moiety L, in some embodiments, is a peptide that is at least five amino acids in length and no longer than about 50 amino acids in length, e.g., about any of 45, 40, 35, 30, 25, 20, 15, 10 amino acids in length. In an embodiment, L consists essentially of, or consists of, (GGGGS)ₙ (SEQ ID NO:8), where n is equal to 3, 4 or 5. In an embodiment, L consists essentially of, or consists of, (GGGGS)₃ (SEQ ID NO:9).

In an embodiment, the ARSB fusion protein comprises, consists essentially of, or consists of the amino acid sequence shown in FIG. 3A (SEQ ID NO:10) or FIG. 3B (SEQ ID NO:11).

In another aspect, the present disclosure provides a polynucleotide (e.g., an isolated polynucleotide) which comprises a nucleotide sequence that encodes an ARSB fusion protein described herein. In an embodiment, the nucleotide sequence is operably linked to a promoter sequence and a polyA signal sequence. In an embodiment, the promoter sequence is identical to, or substantially identical to, one of the promoter sequences in FIG. 4. In an embodiment, the polyA signal sequence is identical to, or substantially identical to, one of the polyA signal sequences shown in FIG. 5. In an embodiment, the ARSB fusion protein consists of the amino acid sequence shown in FIG. 6A and the polynucleotide comprises, consists essentially or, or consists of the nucleotide sequence shown in FIG. 6B.

In yet another aspect, the present disclosure provides a mammalian cell (e.g., a mouse cell, a Chinese hamster ovary (CHO) cell, a monkey cell, a human cell (e.g., an RPE cell) that is genetically modified to express and secrete an ARSB fusion protein described herein. In an embodiment, the mammalian cell is genetically modified by transfection with a polynucleotide described herein (e.g., an expression vector) which encodes the ARSB fusion protein. In an embodiment, the mammalian cell is derived from an RPE cell (e.g., an ARPE-19 cell) by transfection with an expression vector consisting essentially of, or consisting of, the nucleotide sequence shown in FIG. 7 (SEQ ID NO:20).

The present disclosure also provides a composition comprising a plurality of genetically modified cells described herein and a method of manufacturing the composition. In an embodiment, the composition comprises a cell culture media or a storage medium. In an embodiment, the composition comprises a polymer solution in which the cells are suspended, e.g., a polymer solution described herein, e.g., comprising alginate and a cell-binding substance, e.g., as defined herein. In an embodiment, the method of manufacturing the composition comprises culturing a plurality of a genetically modified cell described herein until a desired number of cultured cells has been produced, and combining the desired number of cultured cells with a cell culture media, a storage medium or a polymer solution.

In yet another aspect, the present disclosure features a device comprising at least one cell-containing compartment which comprises a genetically modified mammalian cell as described herein or a plurality of such cells. The device is configured to shield the cell(s) from the recipient's immune system and mitigate the foreign body response (FBR) (as defined herein) to the implanted device. In an embodiment, the surface of the device comprises a compound or polymer that mitigates the FBR (as defined herein) to the device (e.g., an afibrotic compound or afibrotic polymer). In an embodiment, an afibrotic polymer comprises a biocompatible, zwitterionic polymer, e.g., as described in WO 2017/218507, WO 2018/140834, or Liu et al., Zwitterionically modified alginates mitigate cellular overgrowth for cell encapsulation, Nature Communications (2019)10:5262. In an embodiment, the compound is a compound of Formula (I):

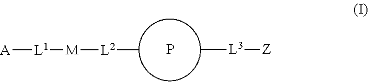

or a pharmaceutically acceptable salt thereof, wherein the variables A, L¹, M, L², P, L³, and Z, as well as related subvariables, are defined herein. In some embodiments, the compound of Formula (I) or a pharmaceutically acceptable salt thereof (e.g., Formulas (I-a), (I-b), (I-b-i), (I-b-ii), (I-c), (I-d), (I-e), (I-f), (II), (II-a), (III), (III-a), (III-b), (III-c), (III-d), (IV-a), (IV-b), (IV-c), (IV-d), or (IV-e)) is a compound described herein, including for example, one of the compounds shown in Table 5 herein, e.g., Compound 100, Compound 101, Compound 102 or Compound 122 shown in Table 5.

In an embodiment, the hydrogel capsules in the hydrogel capsule population are sphere-like or spherical in shape. In an embodiment, the hydrogel capsules have a mean capsule diameter of about 500 μm to about 5000 μm (e.g., about 500 μm to about 4000 μm, about 500 μm to about 3000 μm, about 500 μm to about 2500 μm, about 500 μm to about 2000 μm, about 500 μm to about 1500 μm, about 500 μm to about 1000 μm, about 1000 μm to about 2500 μm). In an embodiment, the hydrogel capsules are not sphere-like or spherical in shape. In an embodiment, each capsule comprises a cell-containing compartment surrounded by a barrier compartment which comprises the ionically cross-linked alginate. In an embodiment, the cross-linked alginate comprises an alginate covalently modified with at least one afibrotic compound, as defined herein. In an embodiment, the cross-linking agent comprises barium ions. In some embodiments, the cell containing compartment encapsulates the live mammalian cells in a first polymer composition comprising an alginate, which is optionally ionically cross-linked (e.g., with barium ions). In some embodiments, the alginate in the first polymer composition is covalently modified with a cell-contacting peptide, as defined herein. In an embodiment, the mean capsule diameter of the hydrogel capsules is 1400 to 2000 μm.

In one aspect, a device of the disclosure is a two-compartment hydrogel capsule (e.g., a microcapsule (less than 1 mm in diameter) or a millicapsule (at least 1 mm in diameter)) in which a cell-containing compartment (e.g., the inner compartment) comprising a plurality of live genetically modified cells described herein (and optionally one or more cell binding substances) is surrounded by a barrier compartment comprising an afibrotic polymer (e.g., the outer compartment, e.g., hydrogel layer). In an embodiment, the afibrotic polymer comprises an afibrotic compound. In an embodiment, the afibrotic compound is a compound of Formula (I).

In another aspect, the present disclosure features a preparation (e.g., a composition) comprising a plurality (at least any of 3, 6, 12, 25, 50 or more) of a cell-containing device described herein, e.g, a preparation of hydrogel capsules encapsulating genetically modified cells, e.g., genetically modified ARPE-19 cells. In some embodiments, the preparation is a pharmaceutically acceptable composition.

In another aspect, the present disclosure features a method of making or manufacturing a device comprising a genetically modified cell described herein. In some embodiments, the method comprises providing the genetically modified cell, or a plurality of such cells, and disposing the cell(s) in an enclosing component, e.g., a cell-containing compartment of the device as described herein. In some embodiments, the enclosing component comprises a flexible polymer (e.g., PLA, PLG, PEG, CMC, or a polysaccharide, e.g., alginate). In some embodiments, the enclosing component comprises an inflexible polymer or metal housing. In some embodiments, the surface of the device is chemically modified, e.g., with a compound of Formula (I) as described herein.

In an embodiment, a device described herein, or a plurality of the device, is combined with a pharmaceutically acceptable excipient to prepare a device preparation or a composition which may be administered to a subject (e.g., into the intraperitoneal cavity) in need of treatment with the ARSB fusion protein produced by the device. In an embodiment, the genetically modified cells are derived from a human cell (e.g., an RPE cell, an ARPE-19 cell) and the device preparation or composition is capable of continuously delivering an effective amount of the ARSB fusion protein to the subject for a sustained time period, e.g., at least any of 3 months, 6 months, one year, two years or longer.

In another aspect, the present disclosure features a method of evaluating a composition, device or device preparation described herein. In some embodiments, the method comprises providing the composition, device or device preparation and evaluating a functional parameter of the composition, device or device preparation. In an embodiment, the functional parameter is the amount of the ARSB fusion protein produced by the cells in the composition, device or device preparation in vitro (e.g., when placed in a suitable culture medium) and/or in vivo (e.g., after implant into a subject, e.g., a non-human subject or a human subject.

In another aspect, the present disclosure features a method of treating a subject in need of therapy with an ARSB fusion protein described herein, e.g., a human subject diagnosed with MPS-6. In an embodiment, the method comprises administering to the subject a device or device preparation comprising a genetically modified cell that expresses and secrets the fusion protein, or a plurality of such cells. In some embodiments, the administering step comprises placing into the subject a pharmaceutically acceptable preparation comprising a plurality of devices, each of which has the ability to produce the ARSB fusion protein. In some embodiments, the implantable element is administered to, placed in, or injected in the peritoneal cavity (e.g., the lesser sac), the omentum, or the subcutaneous fat of a subject. In an embodiment, the method further comprises measuring the amount of the ARSB fusion protein present in a tissue sample removed from the subject, e.g., in plasma separated from a blood sample. In an embodiment, the tissue sample is removed at 15, 30, 60 or 120 days after administration, implantation, or placement of the device or device preparation.

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIG. 1 shows the amino acid sequence of precursor human ARSB (SEQ ID NO: 1).

FIG. 2 shows the amino acid sequence of precursor human serum albumin (SEQ ID NO:2), with the signal peptide underlined.

FIGS. 3A-3B show the amino acid sequence of exemplary ARSB fusion proteins of the disclosure, the fusion protein in FIG. 3A (SEQ ID NO:10) comprising a parental anti-HSA sdAb sequence and the fusion protein in FIG. 3B (SEQ ID NO:11) comprising a humanized variant of the anti-HSA sdAb sequence shown in FIG. 3A, with underlining indicating the anti-HSA sdAb sequence, italics font in each sequence indicating the linker peptide, and bold font indicating the human mature ARSB sequence.

FIGS. 4A-4C show exemplary promoter sequences that are useful in an expression construct for ARSB fusion proteins of the disclosure: pCAG promoter sequence (FIG. 4A, SEQ ID NO:12); EF1α promoter sequence (FIG. 4B, SEQ ID NO:13); and EFS promoter sequence (FIG. 4C, SEQ ID NO:14).

FIGS. 5A-5C show exemplary polyA signal sequences that are useful in an expression construct for ARSB fusion proteins of the disclosure: rBG poly A signal sequence (FIG. 5A, SEQ ID NO:15; SV40 late poly A signal sequence (FIG. 5B, SEQ ID NO:16) and BGH poly A signal sequence (FIG. 5C, SEQ ID NO: 17).

FIGS. 6A-6B show the amino acid and nucleotide coding sequences (SEQ ID NO: 18 and SEQ ID NO:19) for an exemplary precursor ARSB fusion protein of the disclosure, with single underlining indicating the consensus VHH signal peptide, double underlining indicating the R28 sdAb, italics indicating the linker peptide, bold font indicating human mature ARSB.

FIG. 7 shows the nucleotide sequence of an exemplary transposon expression vector (SEQ ID NO:20) useful for genetically modifying human cells to express the ARSB fusion protein shown in FIG. 6A.

FIGS. 8A-8B illustrate evaluating the ARSB activity of ARSB fusion proteins, with FIG. 8A showing a standard curve prepared using a non-fusion recombinant human ARSB (galsulfase) and FIG. 8B comparing the ARSB activity of fusion proteins with an anti-HSA sdAb either N-terminal or C-terminal to the ARSB amino acid sequence.

FIG. 9 shows that the activity of the native and recombinant HL-hARSB fusion proteins exhibit similar biochemical characteristics as measured through ability to decrease the amount of chondroitin sulfate/dermatan sulfate (CS/DS) compared to untreated controls.

FIGS. 10A-10C show that the total hARSB activity in each of the liver (FIG. 10A), heart (FIG. 10B), and spleen (FIG. 10C) is highest in mice treated with hydrogel capsules comprising cells engineered to express the HL-hARSB enzyme.

FIGS. 11A-11C show that the total CS/DS activity in each of the liver (FIG. 11A), heart (FIG. 11B), and spleen (FIG. 11C) is comparable between the HL-hARSB and galsulfase controls in all tested conditions.

### DETAILED DESCRIPTION

The present disclosure features ARSB fusion proteins that comprise an HSA-binding domain located N-terminal to an enzymatic domain comprising a mammalian ARSB protein, mammalian cells (e.g., human RPE cells) genetically modified to express and secrete these ARSB fusion proteins, as well as compositions and devices comprising the genetically modified cells. In some embodiments, the devices comprise a cell-containing compartment which includes a cell binding substance as well as the genetically modified cells. In some embodiments, the devices are configured to mitigate the FBR when placed inside a subject, e.g., a human subject. In some embodiments, the fusion proteins, genetically modified cells, compositions, and devices are useful for the treatment of MPS-6. g.

### Abbreviations and Definitions

Throughout the detailed description and examples of the disclosure the following abbreviations will be used.
CS chondroitin sulfate
DS dermatan sulfate
GAG glycosaminoglycan
MPS mucopolysaccharidoses

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About” or “approximately” when used herein to modify a numerically defined parameter (e.g., amount of a fusion protein secreted by an engineered cell, a physical description of a device (e.g., hydrogel capsule) such as diameter, sphericity, number of cells encapsulated therein, the number of devices in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a hydrogel capsule defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer,” “administering,” or “administration,” as used herein, refer to implanting, absorbing, ingesting, injecting, placing, or otherwise introducing into a subject, an entity described herein (e.g., a device or a preparation of devices), or providing such an entity to a subject for administration.

“Afibrotic”, as used herein, means a compound or material that mitigates the foreign body response (FBR). For example, the amount of FBR in a biological tissue that is induced by implant into that tissue of a device (e.g., hydrogel capsule) comprising an afibrotic compound (e.g., a hydrogel capsule comprising a polymer covalently modified with a compound listed in Table 5) is lower than the FBR induced by implantation of an afibrotic-null reference device, i.e., a device that lacks any afibrotic compound, but is of substantially the same composition (e.g., same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

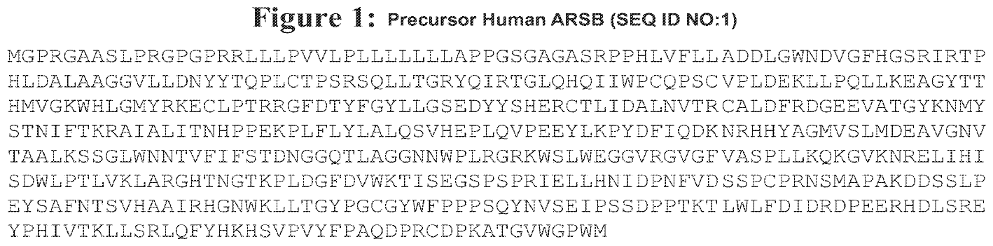

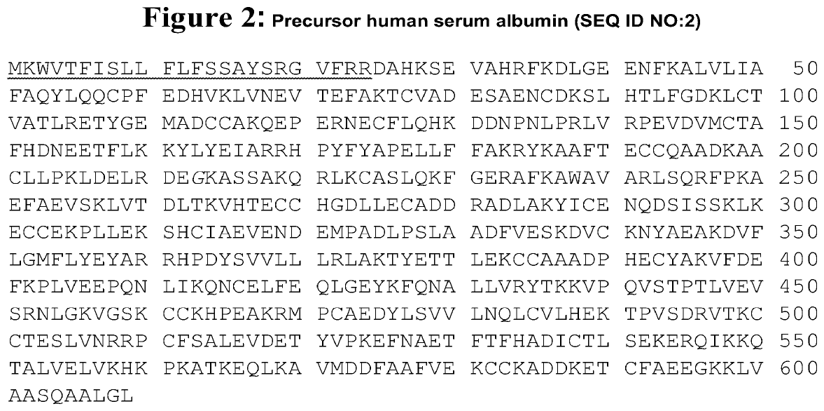

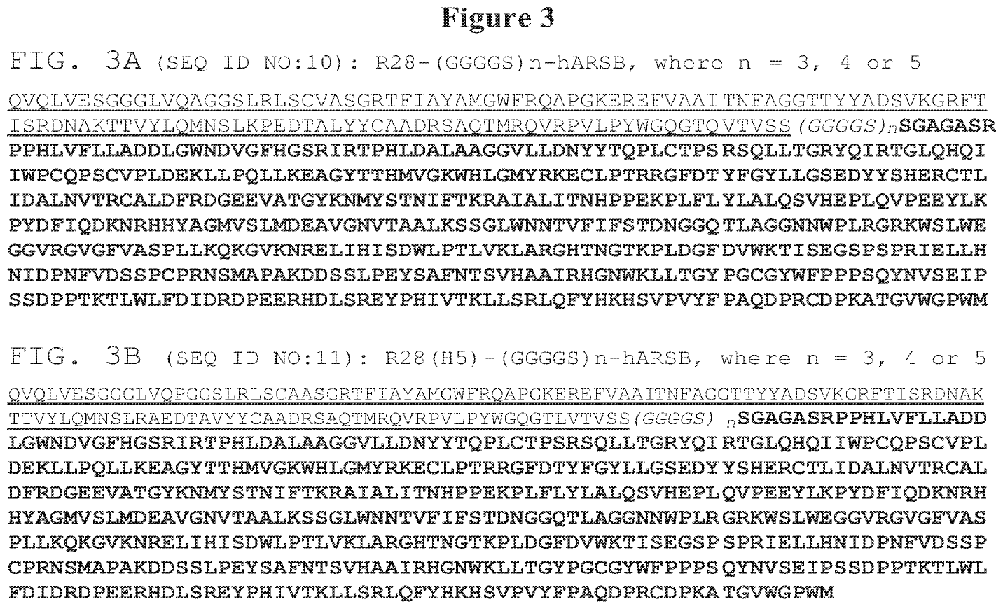

“Arylsulfatase B protein” and “ARSB protein” may be used interchangeably herein and refer to a protein comprising the amino acid sequence of a mature, wild-type mammalian ARSB or any fragment, mutant, variant or derivative thereof that has enzyme activity (e.g., sulfatase activity) that is within 80-120%, 85-115%, 90-110% or 95-105% of the corresponding wild-type mammalian mature ARSB protein, as measured by an ARSB activity assay described herein. ARSB hydrolyses sulfates in the body by metabolizing the sulfate moiety of dermatan sulfate and chondroitin sulfate. The wild-type human ARSB gene encodes a 533 amino acid precursor polypeptide, of which the N-terminal 36 or 38 amino acids constitute a signal peptide. The amino acid sequence for wild-type human precursor ARSB is shown in FIG. 1 (SEQ ID NO:1). In an embodiment, the mature human ARSB sequence is amino acids 37 to 533 of FIG. 1. In another embodiment, the mature human ARSB sequence is amino acids 39 to 533 of FIG. 1. In some embodiments, the term “ARSB protein” refers to a polypeptide comprising the wild-type mature amino acid sequence, and optionally preceded by the ARSB signal peptide or by a signal peptide for a different secretory protein, e.g., a protein secreted by hurnan cells, e.g., ARPE-19 cells.

“Cell,” as used herein, refers to an engineered cell (e.g., a genetically modified cell), or a cell that is not engineered. In an embodiment, a cell is an immortalized cell, or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). In an embodiment, the CBP is any of the CBPs described in international patent publication WO2020069429. In an embodiment, the CBP is a linear peptide comprising RGD (SEQ ID NO:56) and is less than 10 amino acids in length. In an embodiment, the CBP is a linear peptide that consists essentially of GRGD (SEQ ID NO:57) or GRGDSP (SEQ ID NO:58).

“CBP-polymer”, as used herein, means a polymer comprising at least one cell-binding peptide molecule covalently attached to the polymer via a linker. In an embodiment, the polymer in a CBP-polymer is a synthetic or naturally-occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the CBP-polymer is any of the CBP-alginates defined in WO2020069429.

“Cell-binding substance (CBS)”, as used herein, means any chemical, biological, or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide, as defined herein or in WO2020069429.

“Conservatively modified variants” or conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. In some embodiments, a conservatively modified variant of an HSA-binding domain sequence does not include substitutions of any amino acids in the CDRs. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 1 below.

TABLE 1
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, an HSA-binding domain or an ARSB domain that consists essentially of a recited amino acid sequence may also include one or more amino acids, including substitutions in the recited amino acid sequence, of one or more amino acid residues, which do not materially affect the relevant biological activity of the HSA-binding domain or the ARSB portion, respectively.

“Derived from”, as used herein with respect to a cell or cells, refers to cells obtained from tissue, cell lines, or cells, which optionally are then cultured, passaged, immortalized, differentiated and/or induced to produce the derived cell(s).

“Device”, as used herein, refers to any implantable object (e.g., a particle, a hydrogel capsule, an implant, a medical device), which contains an engineered cell or cells (e.g., live cells) capable of expressing and secreting a fusion protein following implant of the device, and has a configuration that supports the viability of the cells by allowing cell nutrients to enter the device.

“Effective amount”, as used herein, refers to an amount of any of the following: genetically-modified cells secreting an ARSB fusion protein, a device preparation producing the fusion protein, number of genetically-modified cells in a device, amount of a CBS and/or afibrotic compound in a device that is sufficient to elicit a desired biological response. In some embodiments, the term “effective amount” refers to the amount of a component of the device (e.g., number of cells in the device, the density of an afibrotic compound disposed on the surface and/or in a barrier compartment of the device, the density of a CBS in the cell-containing compartment.

In an embodiment, the desired biological response is an increase in levels of the ARSB protein in a tissue sample removed from a subject treated with (e.g., implanted with) the genetically modified cells, a device or a device preparation containing such cells. As will be appreciated by those of ordinary skill in this art, the effective amount may vary depending on such factors as the desired biological endpoint, the pharmacokinetics of the secreted ARSB fusion protein, composition or device, the condition being treated, the mode of administration, and the age and health of the subject. An effective amount encompasses therapeutic and prophylactic treatment.

In an embodiment, an effective amount of a compound of Formula (I) disposed on or in a device is an amount that reduces the FBR to the implanted device compared to a reference device, e.g., reduces fibrosis or amount of fibrotic tissue on or near the implanted device.

In an embodiment, an effective amount of a CBS disposed with engineered cells in a cell-containing compartment is an amount that enhances the viability of the cells (e.g., number of live cells) compared to a reference device and/or increases the production of the ARSB fusion protein by the cells (e.g., increased levels of the fusion protein in plasma of a subject implanted with the device) compared to a reference device. An effective amount of a device, composition or component (e.g., afibrotic compound, CBS, engineered cells) may be determined by any technique known in the art of described herein.

“Engineered cell” or “genetically-modified cell,” as used herein, is a mammalian cell (e.g., a human cell, e.g., an RPE cell, a cell derived from a cell line, (e.g., ARPE-19 or other cell line), a stem cell, a cell differentiated from an iPSC) having at least one non-naturally occurring alteration, and typically comprises an exogenous nucleotide sequence (e.g., a vector or an altered chromosomal sequence), encoding an ARSB fusion protein described herein. In an embodiment, the exogenous nucleotide sequence is chromosomal (e.g., the exogenous sequence is disposed in endogenous chromosomal sequence) or is extra chromosomal (e.g., a non-integrated expression vector). In an embodiment, the exogenous nucleotide sequence in a genetically modified cell comprises a codon optimized coding sequence for one of both of the AB and ARSB domains in the fusion protein that achieves higher expression by the modified cell of the fusion protein compared to a naturally-occurring coding sequence for each of these domains. The codon optimized sequence may be generated using a commercially available algorithm, e.g., GeneOptimizer (ThermoFisher Scientific), OptimumGene^{T}M (GenScript, Piscataway, NJ USA), GeneGPS® (ATUM, Newark, CA USA), or Java Codon Adaptation Tool (JCat, www.jcat.de, Grote, A. et al., Nucleic Acids Research, Vol 33, Issue suppl_2, pp. W526-W531 (2005). In an embodiment, the cell is also genetically modified to reduce or eliminate expression of one or more proteins naturally expressed by the parental cell. In an embodiment, a genetically modified cell (e.g., modified RPE cell, a modified ARPE-19 cell) is cultured from a population of stably-transfected cells, or from a monoclonal cell line.

“An “exogenous nucleotide sequence,” as used herein, is a nucleotide sequence that does not occur naturally in a subject cell.

An “exogenous polypeptide,” as used herein, is a polypeptide that does not occur naturally in a subject cell, e.g., engineered cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Expression vector”, as used herein, refers to a recombinant polynucleotide comprising one or more expression constructs encoding one or more proteins to be expressed. Each expression construct contains expression control sequences operatively linked to one or more nucleotide sequences to be expressed. An expression vector comprises sufficient cis-acting elements for expression; other elements for expression can be supplied by the host cell or in an in vitro expression system. The vector may comprise additional sequence elements used for the expression of and/or the integration of the expression cassette(s) into the genome of a mammalian cell. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vectors suitable for use in engineering mammalian cells to express any of the fusion proteins described herein may also contain a nucleotide sequence encoding a marker for selection of cells that contain such a vector. Examples of a suitable marker are genes that encode resistance to antibiotics, such as ampicillin, chloramphenicol, kanamycin, nourseothricin, or zeocin.

“High molecular weight alginate” or “HMW-Alg”, as used herein, means an alginate with an approximate molecular weight of 150 kDa-250 kDa.

“Low molecular weight alginate” or “LMS-Alg” as used herein, means an alginate with an approximate molecular weight of <75 kDa.

“Medium molecular weight alginate” or “MMW-Alg” as used herein, means an alginate with an approximate molecular weight of 75 kDa to 150 kDa.

“MPS-6 patient” as used herein, refers to an individual who has been diagnosed with or suspected of having MPS-6 disease. In an embodiment, an MPS-6 patient has a mutated ARSB gene. The patient may be diagnosed using any method known in the art, including one or more of the clinical, biochemical and genetic methods for diagnosing MPS-6 described in Vairo et al., The Application of Clinical Genetics Vol. 8, pp. 245-255 (2015).

“Peptide”, as used herein, is a polypeptide of less than 50 amino acids, typically, less than 25 amino acids.

“PolyA signal”, as used herein, refers to any continuous sequence that terminates transcription of a coding sequence into RNA and directs addition of a polyA tail onto the RNA. Examples of polyA signals are the rabbit binding globulin (rBG) polyA signal, the SV40 late poly A signal, the SV50 polyA signal, the bovine growth hormone (BGH) poly A signal, the human growth hormone (HGH) polyA signal and synthetic polyA signals known in the art.

“Polymer composition”, as used herein, is a composition (e.g., a solution, mixture) comprising one or more polymers. As a class, “polymers' includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, is a polymer comprising amino acid residues linked through peptide bonds and having at least two, and in some embodiments, at least 10, 50, 75, 100, 150 or 200 amino acid residues.

“Prevention,” “prevent,” and “preventing”, as used herein, refer to a treatment that comprises administering or applying an ARSB fusion protein described herein, e.g., administering a composition of devices encapsulating modified cells expressing the fusion protein (e.g., as described herein), prior to the onset of one or more symptoms of MPS-6 to preclude the physical manifestation of the symptom(s). In some embodiments, “prevention,” “prevent,” and “preventing” require that signs or symptoms of MPS-6 have not yet developed or have not yet been observed. In some embodiments, treatment comprises prevention and in other embodiments it does not.

“Promoter sequence”, as used herein refers to a nucleotide sequence that is capable of driving expression in a mammalian cell, e.g., a human cell, e.g., a cell derived from a mammalian cell line, e.g., an RPE cell line, an iPSC cell line. In some embodiments, e.g., for driving expression of an ARSB fusion protein described herein, the promoter sequence is from a strong mammalian promoter, e.g., a human promoter sequence. Non-limiting examples of strong promoters for use in expression cassettes described herein include the EF1A promoter, CAG promoter, PGK (phosphoglycerate kinase) promoter and the ACTB (human beta-actin) promoter. In an embodiment, a promoter sequence useful for driving expression of a ST protein described herein may be from a medium-strength promoter, e.g., the EFS promoter sequence, which is a shortened form of the EF1A promoter sequence.

“RPE cell” as used herein refers to a cell having one or more of the following characteristics: a) it comprises a retinal pigment epithelial cell (RPE) (e.g., cultured using the ARPE-19 cell line (ATCC® CRL-2302™)) or a cell derived or modified therefrom, e.g., by stably transfecting cells cultured from the ARPE-19 cell line with an exogenous sequence that encodes an ARSB fusion protein), a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; or iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE described herein is genetically modified, e.g., to have a new property, e.g., the cell is modified to express and secrete a fusion protein described herein. In other embodiments, an RPE cell is not genetically modified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455 A1. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical” as used herein, mean a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Subject” as used herein refers to a human or non-human animal. In an embodiment, the subject is a human (i.e., a male or female) of any age group, e.g., a pediatric human subject (e.g., infant, child, adolescent) or adult human subject (e.g., young adult, middle-aged adult, or senior adult)). In an embodiment, the subject is a non-human animal, for example, a mammal (e.g., a mouse, a dog, a primate (e.g., a cynomolgus monkey or a rhesus monkey). In an embodiment, the subject is a commercially relevant mammal (e.g., cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Treatment,” “treat,” and “treating” as used herein refers to one or more of reducing, reversing, alleviating, delaying the onset of, or inhibiting the progress of one or more of a symptom, manifestation, or underlying cause, of MPS-6. In an embodiment, treating comprises reducing, reversing, alleviating, delaying the onset of, or inhibiting the progress of a symptom associated with MPS-6. In some embodiments, “treatment,” “treat,” and “treating” require that signs or symptoms associated with MPS-6 have developed or have been observed. In other embodiments, treatment may be administered in the absence of signs or symptoms of disease, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In some embodiments, treatment comprises prevention and in other embodiments it does not.

“Wild-type” (wt) refers to the natural form, including sequence, of a polynucleotide, polypeptide or protein in a species. A wild-type form is distinguished from a mutant form of a polynucleotide, polypeptide or protein arising from genetic mutation(s).

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁—C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents, e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (Cs), pentadienyl (Cs), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (C₁), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful for preparing devices in the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable device, e.g., a particle, a hydrogel capsule. The connection represented by “ [symbol] ” may refer to direct attachment to the entity, e.g., a polymer or an implantable element, may refer to linkage to the entity through an attachment group. An “attachment group,” as described herein, refers to a moiety for linkage of a compound of Formula (I) to an entity (e.g., a polymer or an implantable element (e.g., a device) as described herein), and may comprise any attachment chemistry known in the art. A listing of exemplary attachment groups is outlined in Bioconjugate Techniques (3^{rd} ed, Greg T. Hermanson, Waltham, MA: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)ₓ—, —OS(O)ₓ—, —N(R^{C})S(O)ₓ, —S(O)ₓN(R^{C})—, —P(R^{F})_{y}—, —Si(OR^{A})₂—, —Si(R^{G})(OR^{A}), —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl. In some embodiments, an attachment group is a cross-linker. In some embodiments, the attachment group is —C(O)(C₁-C₆¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### Features of ARSB Fusion Proteins

ARSB fusion proteins of the disclosure comprise an HSA-binding domain (AB) located upstream of an amino acid sequence for a mature mammalian ARSB protein with ARSB enzymatic activity (ARSB). In some embodiments, a linker moiety (e.g., a linker peptide) is located between the AB and ARSB domains.

### HSA-Binding Domain AB

The serum half-life of the ARSB fusion protein is longer than the serum half-life of an otherwise identical fusion protein without the AB domain. This half-life extension is largely due to binding of the fusion protein via AB to HSA. Mature human serum albumin (HSA) is a monomeric protein with 585 amino acids (GenBank Accession No. AAA98797.1), and contains three partially overlapping domains: DI (amino acids 25-221 of FIG. 1); DII (amino acids 213-409 of FIG. 1); and DIII (amino acids 405-609 of FIG. 1) (Dockal, M., et al., J. Biol. Chem., Vol. 274, No. 41, pp. 29303-29310 (1999). The long half-life of albumin in blood is mainly driven by two characteristics: (i) the large size (65 kDa) of albumin limits its glomerular filtration and (ii) albumin binds to FcRn at low pH (pH 6), which protects albumin from degradation in the lysosomes after passive endocytosis in endothelial and epithelial cells, by recycling from early endosome back to the extracellular environment. In an embodiment, the AB domain confers upon the ARSB fusion protein a serum half-life in man (expressed as t½-beta) that is more than any of 6 hours, 12 hours, 24 hours, 72 hours, one week, two weeks or up to the half-life of HSA in man (estimated to be around 19 days).

In some embodiments, the AB domain is specific for serum albumin, i.e., it does not substantially bind to any non-albumin proteins. In some embodiments, the fusion protein does not substantially inhibit binding of FcRn to HSA, the binding site of which is in DIII. In an embodiment, less than about 20%, 15%, 10%, 5% or 1% inhibition of FcRn binding to HSA occurs in the presence of a fusion protein of the disclosure, which may be determined by any competition binding assay known in the art, e.g., the SPR competition binding assay described in WO 2019/204925 at [0095] or in Example 3 of US 2019/0367596A1. The potential for interference with FcRn binding can be reduced by deriving the AB domain from an anti-HSA antibody that does not binds to DIII. In an embodiment, the fusion protein binds via the AB domain to DI of HSA. In an embodiment, the fusion protein cross-competes via AB for HSA binding with a sdAb that consists of the amino acid sequence for a single domain antibody (sdAb) described in WO2019/204925, i.e., R11, R28, M75 or M79.

In some embodiments, AB (as part of the fusion protein) also binds to (e.g., cross-reacts with) serum albumin from one, two, three, four or more other mammalian species, e.g., any combination of two, three, four or more of mouse, rat, guinea pig, hamster, rabbit, cat, dog, pig, sheep, horse, cow and monkey (e.g., rhesus and/or cynomolgus). In an embodiment, the fusion protein binds via the AB domain to serum albumin from at least mouse, rat, monkey (rhesus or cynomolgus) and human. In an embodiment, the fusion protein binds via the AB domain to serum albumin from at least mouse, dog and human.

In some embodiments, the AB domain (as part of the fusion protein) binds to HSA, and optionally to at least one other mammalian serum albumin, with a desired affinity within a pH range of between about 5.0 or about 5.5 up to about 7.4 In an embodiment, the desired affinity is a dissociation constant (K_{D}) ranging from any of about 0.1 nM to about 1,000 nM, about 0.5 nM to about 500 nM, about 1 nM to about 250 nM, about 5 nM to about 50 nM, about 10 nM to about 25 nM, or about 0.5 nM to about 1 nM. In an embodiment, the affinity is determined by surface plasmon resonance (SPR) at 25° C. and a pH range of about 5.5 to about 7.4. In an embodiment, the affinity of the fusion protein for serum albumin from mouse, rat and monkey is similar to the affinity for HSA, e.g., within 70% to 130%, 80% to 120%, or 90% to 110% of the K_{D} for HSA.

An exemplary SPR assay for measuring K_{D} of the anti-HSA antibody and the fusion protein is described in WO 2019/204925 at [0092]. Another exemplary SPR assay for measuring affinities within the pH range of 5.0 to 7.4 is described in US 2019/0367597A1. The K_{D} of the anti-HSA antibody and the fusion protein for HSA and mammalian orthologues may also be determined substantially as described in WO 2019/246003 at [0994]-[0995].

The AB domain of an ARSB fusion protein of the disclosure comprises the amino acid sequence of a heavy chain variable region (HCVR) of an anti-HSA antibody. To facilitate expression of the fusion protein by genetically-modified cells, in some embodiments, the AB domain has a molecular weight of less than about any of 75 kDa, 50 kDa or 25 kDa. The AB domain may be derived from any anti-HSA antibody molecule known in the art, including conventional 4-chain antibodies, antigen-binding fragments, Fab, Fab′, F(ab′)₂, Fv (double-chain and single-chain (scFv)), minibody, diabody and sdAb. In an embodiment, AB consists essentially of, or consists of, the HCVR amino acid sequence of a sdAb.

In an embodiment, the AB domain comprises the set of three CDR amino acid sequences 10 in one of the anti-HSA sdAbs described in Table I of WO 2006/122787. In an embodiment, AB consists essentially of, or consists of, the amino acid sequence in one of the anti-HSA sdAbs described in Table II of WO 2006/122787 (e.g., Alb-1), or in one of the humanized variants of Alb-1 described in Table III of WO 2006/122787 (e.g., Alb-8). In an embodiment, AB consists essentially of, or consists of, any of the Alb-23 sequences described in WO 2012/175400 (e.g., Alb-23D). In an embodiment, an ARSB fusion protein of the present disclosure cross-competes for binding to HSA with any of the anti-HSA sdAbs described in WO 2006/122787 (e.g., Alb-8) or in WO 2012/175400 (e.g., Alb-23D). In an embodiment, the ARSB fusion protein does not comprise the amino acid seuence for Alb-1. In an embodiment, the ARSB fusion protein does not comprise the amino acid sequence for Alb-8.

In an embodiment, the AB comprises (e.g., or consists of) the amino acid sequence of one of the following sequences, e.g., described in Table II of WO 2006/122787:

```
(i) PMP6A8(ALB2):
(SEQ ID NO: 62)
AVQLVESGGGLVQGGGSLRLACAASERIFDLNLMGWYRQGPGNERELVATCITVG
DSTNYADSVKGRFTISMDYTKQTVYLHMNSLRPEDTGLYYCKIRRTWHSELWGQG
TQVTVSS
(ii) PMP6B4-
(SEQ ID NO: 63)
EVQLVESGGGLVQEGGSLRLACAASERIWDINLLGWYRQGPGNERELVATITVGDSTSY
ADSVKGRFTISRDYDKNTLYLQMNSLRPEDTGLYYCKIRRTWHSELWGQGTQVTVSS
(iii) PMP6A6(ALB1)-
(SEQ ID NO: 64)
AVQLVESGGGLVQPGNSLRLSCAASGFTFRSFGMSWVRQAPGKEPEWVSSISGSGSDTL
YADSVKGRFTISRDNAKTTLYLQMNSLKPEDTA VYYCTIGGSLSRSSQGTQVTVSS
(iv) PMP6C1
(SEQ ID NO: 65)
AVQLVDSGGGLVQPGGSLRLSCAASGFSFGSFGMSWVRQYPGKEPEWVSSINGRGDDT
RYADSVKGRFSISRDNAKNTLYLQMNSLKPEDTA EYYCTIGRSVSRSRTQGTQVTVSS
(v) PMP6G8-
(SEQ ID NO: 66)
AVQLVESGGGLVQPGGSLRLTCTASGFTFRSFGMSWVRQAPGKDQEWVSAISADSS
TKNYADSVKGRFTISRDNAKKMLYLEMNSLKPEDTAVYYCVIGRGSPSSPGTQVTVSS
(vi) PMP6AS-
(SEQ ID NO: 67)
QVQLAESGGGLVQPGGSLRLTCTASGFTFGSFGMSWVRQAPGEGLEWVSAISADSSDK
RYADSVKGRFTISRDNAKKMLYLEMNSLKSEDTA VYYCVIGRGSPASQGTQVTVSS
(vii) PMP6G7-
  (SEQ ID NO: 68)
QVQL VESGGGLVQPGGSLRLSCAASGFTFSNYWMYWVRVAPGKGLERISRDISTGG
GYSYYADSVKGRFTISRDNAKNTLYLQMNSLKPEDTALYYCAKDREAQVDTLDFDY
RGQGTQVTVSS.
In an embodiment, the AB comprises (e.g., or consists of)
the amino acid sequence of one of the following sequences,
e.g., described in Table III of WO 2006/122787:
(i) ALB3 (ALB1 HUM1):
(SEQ ID NO: 69)
EVQLVESGGGLVQPGGSLRLSCAASGFTFRSFGMSWVRQAPGKEPEWVSSISGSGS
DTLYADSVKGRFTISRDNAKTTLYLQMNSLKPEDTAVYYCTIGGSLSRSSQGTQVT
VSS
(ii) ALB4 (ALB1 HUM2):
(SEQ ID NO: 70)
EVQLVESGGGLVQPGGSLRLSCAASGFTFSSFGMSWVRQAPGKEPEWVSSISGSGSDTL
YADSVKGRFTISRDNAKTTLYLQMNSLKPEDTAVYYCTIGGSLSRSSQGTQVTVSS
(iii) ALB5 (ALB1 HUM3):
(SEQ ID NO: 71)
EVQLVESGGGLVQPGGSLRLSCAASGFTFRSFGMSWVRQAPGKGLEWVSSISGSGSDTL
YADSVKGRFTISRDNAKTTLYLQMNSLKPEDTA VYYCTIGGSLSRSSQGTQVTVSS
(iv) ALB6 (ALB1 HUM1):
(SEQ ID NO: 72)
EVQLVESGGGLVQPGNSLRLSCAASGFTFRSFGMSWVRQAPGKGLEWVSSISGSGSDTL
YADSVKGRFTISRDNAKTTLYLQMNSLKPEDTAVYYCTIGGSLSRSSQGTLVTVSS
(v) ALB7 (ALB1 HUM2):
(SEQ ID NO: 73)
EVQLVESGGGLVQPGNSLRLSCAASGFTFRSFGMSWVRQAPGKGLEWVSSISGSGS
DTLYADSVKGRFTISRDNAKTTLYLQMNSLRPEDTAVYYCTIGGSLSRSSQGTLVTV
SS
(vi) ALB8 (ALB1 HUM3):
(SEQ ID NO: 74)
EVQLVESGGGLVQPGNSLRLSCAASGFTFSSFGMSWVRQAPGKGLEWVSSISGSGSDTL
YADSVKGRFTISRDNAKTTLYLQMNSLRPEDTAVYYCTIGGSLSRSSQGTLVTVSS
(vii) ALB9 (ALB1 HUM4):
(SEQ ID NO: 75)
EVQL VESGGGLVQPGNSLRLSCAASGFTFSSFGMSWVRQAPGKGLEWVSSISGSGS
DTLYADSVKGRFTISRDNAKNTLYLQMNSLRPEDTAVYYCTIGGSLSRSSQGTL VT
VSS
(viii) ALB10 (ALB1 HUM5):
  (SEQ ID NO: 76)
EVQL VESGGGLVQPGNSLRLSCAASGFTFSSFGMSWVRQAPGKGLEWVSSISGSGS
DTLYADSVKGRFTISRDNAKNTLYLQMNSLRPEDTAVYYCTIGGSLSRSGQGTL VT
VSS.
```

In another embodiment, the AB domain consists essentially of, or consists of, the amino acid sequence of an albumin binding domain described in Table 7 of WO 2019/246003, e.g., LAEAKVLANRELDKYGVSDYYKNLINNAKTVEGVKALIDEILAALP (SEQ ID NO:21), which is described in WO 2019/246003 as an albumin binding domain having a K_{D} to HSA of about 1.2 nM. In an embodiment, the AB domain of an ARSB fusion protein comprises the three CDRs of the anti-HSA P367 antibody described in Table 14 of WO 2019/2460003. In an embodiment, AB consists essentially of, or consists of, the amino acid sequence of the anti-HSA P367 antibody or its humanized variant P494, each of which is listed in Table 14 of WO 2019/2460003. In an embodiment, an ARSB fusion protein of the present disclosure cross-competes for binding to HSA with the P494 sdAb described in WO 2019/2460003.

In another embodiment, the AB domain comprises a set of three heavy chain CDR amino acid sequences in one of the anti-HSA sdAbs described in Table 5 of WO 2021/119551. In an embodiment, AB consists essentially of, or consists of, the VH amino acid sequence in one of the anti-HSA sdAbs described in Table 5 of WO 2021/119551. In an embodiment, an ARSB fusion protein of the present disclosure cross-competes for binding to HSA with one or more of the sdAbs described in Table 5 of WO 2021/119551.

In another embodiment, the AB domain comprises a set of the three CDR amino acid sequences that are in the T0235002C06 sdAb described in Table B of US 20190367597A1. In an embodiment, AB consists essentially of, or consists of, the amino acid sequence of T0235002C06 described in Table B of US 2019/0367597A1. In an ernbodirnent, an ARSB fusion protein of the present disclosure cross-competes for binding to HSA with the T0235002C06 sdAb described in Table B of US 20190367597A1.

In another embodiment, the AB domain comprises a set of the three CDR amino acid sequences that are in the T0235005D04 sdAb described in Table B of US 20190367596A1. In an embodiment, AB consists essentially of, or consists of, the amino acid sequence of T0235005D04 described in Table B of US 2019/0367596A1. In an embodiment, an ARSB fusion protein of the present disclosure cross-competes for binding to HSA with the T0235002D04 sdAb described in Table B of US 20190367596A1.

In yet another embodiment, the AB domain comprises a set of the three CDR amino acid sequences that are in the T0235005G01 sdAb or T023500043 sdAb described in Table B of US 20190367598A1. In an embodiment, AB consists essentially of, or consists of, the amino acid sequence of T0235005G01 or T023500043 described in Table B of US 20190367598A1. In an embodiment, an ARSB fusion protein of the present disclosure cross-competes for binding to HSA with the T0235005G01 or T023500043 sdAbs described in Table B of US 20190367598A1.

In an embodiment, the AB domain comprises a set of the three CDR amino acid sequences that are in the R28, R11, M75 or M79 sdAbs described in WO 2019/204925. These CDR sequences are set forth in Table 2A below.

TABLE 2A
Exemplary CDR amino acid sequences
for the AB domain.

| sdAb | CDR1 | CDR2 | CDR3 |
|---|---|---|---|
| R11 | GFLLRSNTM | IRPSGLT | HHTRPPFQRDS |
| | (SEQ ID | (SEQ ID NO: 23) | (SEQ ID NO: 24) |
| | NO: 22) | | or |
| | | | ATRPPFORDS |
| | | | (SEQ ID NO: 25) |
| R28 | GRTFIAYA | ITNFAGGTT | AADRSAQTMRQVRPVLPY |
| | (SEQ ID | (SEQ ID NO: 4) | (SEQ ID NO: 5) |
| | NO: 3) | | |
| M75 | GRTFDNYVM | ISGSGSIT | AAGSRRTYYREPKFYPS |
| | (SEQ ID | (SEQ ID NO: 27) | (SEQ ID NO: 28) |
| | NO: 26) | | |
| M79 | GSTESSSSV | ITSGGST | NVAGRNWVPISRYSPGPY |
| | (SEQ ID | (SEQ ID NO: 30) | (SEQ ID NO: 31) |
| | NO: 29) | | of |
| | | | AVAGRNWVPISRYSPGPY |
| | | | (SEQ ID NO: 32) |

In an embodiment, AB consists essentially of, or consists of, the amino acid sequence of the R28, R11, M75 or M79 sdAbs described in WO 2019/204925. In an embodiment, AB consists essentially of, or consists of, the amino acid sequence of one of the humanized variants of R28, R11, M75 or M79 described in WO 2019/204925. The amino acid sequences of the parental and humanized variants of R28, R11, M75 and M79 are shown in the SEQUENCES Table on pages 46-48 of WO 2019/204925. In an embodiment, AB consists essentially of, or consists of, an amino acid sequence selected from the parental and humanized sequences shown in Table 2B herein below. In an embodiment, AB consists essentially of, or consists of, the parental or humanized amino acid sequence of R28 shown in Table 2B below.

TABLE 2B
Exemplary amino acid sequences for the AB domain.

| sdAb | Parental Sequence | Exemplary Humanized Sequence |
|---|---|---|
| R11 | QVKLEESGGGLVQAGGSLRLSCVGPGEL | QVQLVESGGGLVQPGGSLRLSCAGPGEL |
| | LRSNTMGWYRQAPGKERELVAFIRPSGL | LRSNTMGWYRQAPGKERELVAFIRPSGL |
| | TNYNDAVQGRFTISRDNAKNTVYLQMNA | TNYNDAVQGRETISRDNAKNTVYLQMNS |
| | LKPEDTAVYYCHTRPPFQRDSWGQGTQV | LRAEDTAVYYCHTRPPFQRDSWGQGTLV |
| | TVSS | TVSS |
| | (SEQ ID NO: 33) | (SEQ ID NO:34) |
| R28 | QVQLVESGGGLVQAGGSLRLSCVASGRT | QVQLVESGGGLVQPGGSLRLSCAASGRT |
| | FIAYAMGWFRQAPGKEREFVAAITNFAG | FIAYAMGWFRQAPGKEREFVAAITNFAG |
| | GTTYYADSVKGRFTISRDNAKTTVYLQM | GTTYYADSVKGRFTISRDNAKTTVYLQM |
| | NSLKPEDTALYYCAADRSAQTMRQVRPV | NSLRAEDTAVYYCAADRSAQTMRQVRPV |
| | LPYWGQGTQVTVSS | LPYWGQGTLVTVSS |
| | (SEQ ID NO : 6) | (SEQ ID NO:7) |
| M75 | QVQLVESGGGFVQAGGSLRLSCAASGRT | EVQLVESGGGLVQPGGSLRLSCAASGRT |
| | FDNYVMAWERQAPGKEREFVASISGSGS | FDNYVMAWFRQAPGKGLEFVSSISGSGS |
| | ITNYANSVKDRFTISRDSAKNAIYLQMN | ITNYANSVKDRFTISRDNSKNTLYLQMN |
| | SLKPEDTALYYCAAGSRRTYYREPKFYP | SLRAEDTAVYYCAAGSRRTYYREPKFYP |
| | SWGQGTQVTVSS | SWGQGTLVTVSS |
| | (SEQ ID NO: 35 ) | (SEQ ID NO: 36) |
| M79 | QVKLEESGGGLVQAGGSLKLSCAASGST | EVQLVESGGGLVQPGGSLRLSCAASGST |
| | FSSSSVGWYRQAPGQQRELVAAITSGGS | FSSSSVGWYRQAPGKGLELVSAITSGGS |
| | TNTADSVKGRFTMSRDNAKNTVYLQMRD | TNTADSVKGRFTISRDNSKNTLYLQMNS |
| | LKPEDTAVYYCNVAGRNWVPISRYSPGP | LRAEDTAVYYCNVAGRNWVPISRYSPGP |
| | YWGQGTQVTVSS | YWGQGTLVTVSS |
| | (SEQ ID NO:37) | (SEQ ID NO:38) |

### Generation of HSA Binding Domain AB

The AB portion of an ARSB fusion protein may be derived from any antibody or antigen binding fragment thereof that has the desired properties described herein. The antibody or antigen binding fragment may be already known in the art or identified by via any approach known in the art.

In some embodiments, AB is derived from a single-domain Ab (sdAb). For example, sdAbs of camelid origin lack light chains and thus their antigen binding sites consist of one domain, termed V_{H}H. sdAb have also been observed in shark and are termed V_{NAR}. Other sdAb may be engineered based on human Ig heavy and light chain sequences. As used herein, the term “sdAb” includes sdAbs directly isolated from V_{H}, V_{H}H, V_{L}, or V_{NAR} reservoir of any origin through phage display or other technologies, recombinantly produced sdAbs, as well as those sdAb generated through further modification of such sdAbs by humanization, affinity maturation, stabilization, solubilization, or other methods of antibody engineering. Also encompassed by the present disclosure are homologues, derivatives, or fragments that retain the antigen-binding function and specificity of the parental sdAb.

To generate V_{H}H sdAbs that bind to HSA, the skilled person may employ any approach known in the art, e.g., substantially as described in WO 2019/204925, to generate and screen a phage-displayed V_{H}H library from the heavy-chain-only antibody repertoire of llama or other camelid immunized with a desired antigen.

An HSA-binding domain derived from a sdAb may include the parental framework regions; alternatively, the parental CDRs may be grafted onto V_{NAR}, V_{H}H, V_{H} or V_{L} framework regions of other sdAbs or onto the framework regions of other types of antibody fragments or antibody-like molecules (Fv, scFv, Fab) of any source (e.g., human) or proteins of similar size and nature onto which CDRs can be grafted (for example, see Nicaise, M. et al, Protein Sci 13:1882-91 2004).

In some embodiments, the amino acid sequence in AB is a humanized version (humanized variant) of the parental variable region. Humanization of an antibody or antibody fragment comprises replacing an amino acid in the sequence with its human counterpart, as found in the human consensus sequence, without loss of antigen-binding ability or specificity; this approach reduces immunogenicity of the antibody or fragment thereof when introduced into human subjects. The parental sequence may be humanized using any suitable method known in the art, for example, but not limited to CDR grafting and veneering.

In the process of CDR grafting, one or more than one of the CDRs defined herein may be fused or grafted to a human variable region (V_{H}, or V_{L}), to another human antibody (IgA, IgD, IgE, IgG, and IgM), to antibody fragment framework regions (Fv, scFv, Fab), or to proteins of similar size and nature onto which CDR can be grafted. CDR grafting is known in the art and is described in at least the following: U.S. Pat. Nos. 6,180,370, 5,693,761, 6,054,297, and European Patent No. 626390.

Veneering, also referred to in the art as “variable region resurfacing”, involves humanizing solvent-exposed positions of the antibody or antibody fragment; thus, buried non-humanized residues, which may be important for CDR conformation, are preserved while the potential for immunological reaction against solvent-exposed regions is minimized. Veneering is known in the art and is described in at least the following: U.S. Pat. Nos. 5,869,619, 5,766,886, and 5,821,123, and European Patent No. 519596.

### Linkers

The ARSB fusion protein may contain a linker between the AB domain and the ARSB domain, The linker should be of sufficient length to allow the linked polypeptides to individually fold into 3-dimensional structures having the desired functional activity, e.g., binding or therapeutic. Also, the linker should not be cleavable by any proteases or other enzymes present in the serum.

In some embodiments, the linker is a peptide linker comprising at least two, three or four amino acids and less than about 30 amino acids, e.g., less than about any of 25, 20, 15 or 10 amino acids. Peptide linkers are known in the art and nonlimiting examples are described herein.

The peptide linker may have a naturally occurring sequence, or a non-naturally occurring sequence. For example, a sequence derived from the hinge region of heavy chain only antibodies may be used as the linker. See, for example, WO1996/34103.

Suitable linker peptides typically include G and/or S residues in various formats, with exemplary linkers including GGGG (SEQ ID NO:39), TGGGG (SEQ ID NO:40), GGSSGGSGSSSGSGGSGSSG (SEQ ID NO:41), (GGSS)n, (SEQ ID NO:42), (GGGGS)n (SEQ ID NO:8), (SGGGG)n, (SEQ ID NO:43) and GGGG(SGGGG)n (SEQ ID NO:62), wherein “n” in each case is generally a whole number from 1 to 7, inclusive, provided that the maximum length of about 30 amino acids is not exceeded. Another exemplary peptide linker is SKPTCPPPELLGGPSVFIFPPK (SEQ ID NO:45).

In an embodiment, the fusion protein comprises a peptide linker with a length of between about 15 and 20 amino acids, or between about 20 and 25 amino acids. In an embodiment, the linker consists essentially of, or consists of: (GGGS)₃ (SEQ ID NO:9); (GGGS)₄ (SEQ ID NO:46); or (GGGS)₅ (SEQ ID NO:47). In an embodiment, the linker consists of (GGGS)₃.

### Genetically-Modified Cells

Any of the ARSB fusion proteins described above may be expressed by a mammalian cell(s) genetically modified to express and secrete the fusion protein. The genetically modified cell(s) may be derived from a variety of different mammalian cell types (e.g., human cells), including epithelial cells, endothelial cells, fibroblast cells, mesenchymal stem cells, keratinocyte cells and stem cells, e.g., embryonic stem cells or induced pluripotent stem cells. Exemplary cell types include the cell types recited in WO 2017/075631. In some embodiments, the cells are derived from a cell-line shown in Table 3 below.

TABLE 3
Exemplary cell lines

| | | Germ | |
|---|---|---|---|
| Cell Line | Cell Type | Layer | Commercial Source |
| ARPE-19 | Epithelial (Retinal) | Ectoderm | ATCC (CRL-2302) |
| BJ | Fibroblast (Foreskin) | Ectoderm | ATCC (CRL-2522) |
| CCD-841- | Epithelial (Colon) | Endoderm | ATCC (CRL-1790) |
| CoN | | | |
| HaCat | Keratinocyte | Ectoderm | Addexbio (T0020001) |
| HHSEC | Endothelial (Hepatic Sinusoidal) | Endoderm | Sciencellonline.com (#5000) |
| Huv-EC-C | Endothelial (Embryonic umbilical) | Mesoderm | ATCC (CRL-1730) |
| MCF-10A | Epithelial (Mammary Gland) | Ectoderm | ATCC (CRL-10317) |
| MRC-5 | Fibroblast (Lung) | Mesoderm | ATCC (CCL-171) |
| MSC, human | Mesenchyme (Bone Marrow) | Mesoderm | ATCC (PCS-500-012) |
| MSC, mouse | Mesenchyme (Bone Marrow) | Mesoderm | Cyagen (MU BMX-01001) |
| WS-1 | Fibroblast (Skin) | Ectoderm | ATCC (CRL-1502) |
| 293F | Epithelial (Embryonic Kidney) | Mesoderm | Thermo Fisher (R790007) |

In an embodiment, any of the genetically modified mammalian cells described herein is derived from an RPE cell, e.g., an ARPE-19 cell. In an embodiment, a genetically modified RPE cell comprises any of the expression cassettes, transposons and polynucleotides described herein.

Cells may be genetically modified to express and secrete a desired ARSB fusion protein using any of a variety of genetic engineering techniques known in the art. For example, a cell may be transfected with an expression vector comprising an exogenous nucleotide sequence(s) encoding the desired fusion protein operably linked to control elements necessary or useful for gene expression, e.g., promoters, ribosomal binding sites, enhancers, polyA signal and the like. In some embodiments, the exogenous nucleotide sequence is part of a transcription unit that is stably integrated into the genome of the parental cell.

In an embodiment, the exogenous sequence includes a nucleotide sequence encoding a secretory signal sequence for the fusion protein. In an embodiment, the signal sequence is from a naturally secreted protein. In an embodiment, the signal sequence is MELGLSWVVLAALLQGVQA (SEQ ID NO:48). In some embodiments, the signal sequence consists essentially of an amino acid sequence shown in Table 4 below.

TABLE 4
Exemplary secretory signal peptide sequences

| Protein | Amino Acid Sequence |
|---|---|
| Albumin | MKWVTFISLLFLFSSAYS |
| | (SEQ ID NO: 49) |
| Kappa Leader | MVLQTQVFISLLLWISGAYG |
| | (SEQ ID NO: 50) |
| Plasminogen activator | MQMSPALTCLVLGLALVFGEGSA |
| inhibitor 1 | (SEQ ID NO: 51) |
| Thrombospondin-1 | MGLAWGLGVLFLMHVCGT |
| | (SEQ ID NO: 52) |
| Fibronectin | MLRGPGPGLLLLAVQCLGTAVPSTGA |
| | SKSKR (SEQ ID NO: 53) |
| Basement membrane- | MGWRAAGALLLALLLHGRLLA |
| specific heparan | (SEQ ID NO: 54) |
| sulfate proteoglycan | |
| core protein (HSPG2) | |
| Agrin | MAGRSHPGPLRPLLPLLVVAACVLPG |
| | AGG (SEQ ID NO: 55) |

The genetically modified mammalian cells for use in devices, compositions and methods described herein, e.g., as a plurality of cells in a hydrogel capsule, may be in various stages of the cell cycle. In some embodiments, at least one cell in the plurality of genetically modified cells is undergoing cell division. Cell division may be measured using any known method in the art, e.g., as described in DeFazio A et al (1987) J Histochem Cytochem 35:571-577 and Dolbeare F et al (1983) Proc Natl Acad Sci USA 80:5573-5577, each of which is incorporated by reference in its entirety. In an embodiment at least 1, 2, 3, 4, 5, 10, or 20% of the cells are undergoing cell division, e.g., as determined by 5-ethynyl-2′deoxyuridine (EdU) assay or 5-bromo-2′-deoxyuridine (BrdU) assay. In some embodiments, cell proliferation is visualized or quantified by microscopy (e.g., fluorescence microscopy (e.g., time-lapse or evaluation of spindle formation) or flow cytometry. In some embodiments, none of the cells in the plurality of genetically modified cells are undergoing cell division and are quiescent. In an embodiment, less than 1, 2, 3, 4, 5, 10, or 20% of the cells are undergoing cell division, 5-ethynyl-2′deoxyuridine (EdU) assay, 5-bromo-2′-deoxyuridine (BrdU) assay, microscopy (e.g., fluorescence microscopy (e.g., time-lapse or evaluation of spindle formation), or flow cytometry.

In an embodiment, at least 50%, 60%, 70%, 80%, 90% or more of the genetically modified cells in the plurality are viable. Cell viability may be measured using any known method in the art, e.g., as described in Riss, T. et al (2013) “Cell Viability Assays” in Assay Guidance Manual (Sittapalam, G. S. et al, eds). For example, cell viability may be measured or quantified by an ATP assay, 5-ethynyl-2′deoxyuridine (EdU) assay, 5-bromo-2′-deoxyuridine (BrdU) assay. In some embodiments, cell viability is visualized or quantified by microscopy (e.g., fluorescence microscopy (e.g., time-lapse or evaluation of spindle formation) or flow cytometry. In an embodiment, at least 80% of the engineered cells in the plurality are viable, e.g., as determined by an ATP assay, a 5-ethynyl-2′deoxyuridine (EdU) assay, a 5-bromo-2′-deoxyuridine (BrdU) assay, microscopy (e.g., fluorescence microscopy (e.g., time-lapse or evaluation of spindle formation), or flow cytometry.

Any of the parameters described herein may be assessed using standard techniques known to one of skill in the art, such as histology, microscopy, and various functional assays.

### Measuring ARSB Activity

The activity of ARSB secreted by genetically modified cells or devices described herein may be measured by any direct or indirect ARSB activity assay known in the art. In an embodiment, ARSB activity may be measured using one or both of the in vitro fluorogenic activity assay and cell-based functional assay described in the Examples below.

### Implantable Cell-Encapsulating Devices

A genetically modified cell described herein or a plurality of such cells may be incorporated into an implantable device for use in providing an ARSB protein to a subject in need thereof, e.g., a human subject diagnosed with MPS-6.

An implantable device of the present disclosure comprises at least one barrier that prevents immune cells from contacting cells contained inside the device. At least a portion of the barrier needs to be sufficiently porous to allow proteins (e.g., the fusion protein) expressed and secreted by the cells to exit the device. A variety of device configurations known in the art are suitable.

The device (e.g., particle) can have any configuration and shape appropriate for supporting the viability and productivity of the contained cells after implant into the intended target location. As non-limiting examples, device shapes may be cylinders, rectangles, disks, ovoids, stellates, or spherical. The device can be comprised of a mesh-like or nested structure. In some embodiments, a device is capable of preventing materials over a certain size from passing through a pore or opening. In some embodiments, a device (e.g., particle) is capable of preventing materials greater than 50 kD, 75 kD, 100 kD, 125 kD, 150 kD, 175 kD, 200 kD, 250 kD, 300 kD, 400 kD, 500 kD, 750 kD, or 1,000 kD from passing through.

The device can comprise a hydrogel capsule, or a plurality of the hydrogel capsule. The hydrogel capsule(s) in the device can have any of a variety of shapes: cylinder, cylinder with hemispherical ends (also known as spherocylinder), disc, noodle (e.g., as described in WO 2015/191547), sphere (e.g., as defined herein), or spheroid (e.g., as defined herein). In an embodiment, the hydrogel capsule(s) in a device are spheres, as defined herein.

In an embodiment, the device is a macroencapsulation device. Nonlimiting examples of macrodevices are described in: WO 2019/068059, WO 2019/169089, U.S. Pat. Nos. 9,526,880, 9,724,430 and 8,278,106; European Patent No. EP742818B1, and Sang, S. and Roy, S., Biotechnol. Bioeng. 113(7):1381-1402 (2016).

In an embodiment, the device is a macrodevice having one or more cell-containing compartments. A device with two or more cell-containing compartments may be configured to produce two or more proteins, e.g., cells expressing the ARSB fusion protein would be placed in one compartment and cells expressing a different protein (e.g., a therapeutic protein that can alleviate one or more symptoms of MPS-6) would be placed in a separate compartment. WO 2018/232027 describes a device with multiple cell-containing compartments formed in a micro-fabricated body and covered by a porous membrane.

In an embodiment, the device is configured as a thin, flexible strand as described in U.S. Pat. No. 10,493,107. This strand comprises a substrate, an inner polymeric coating surrounding the substrate and an outer hydrogel coating surrounding the inner polymeric coating. The protein-expressing cells are positioned in the outer coating.

In some embodiments, a device (e.g., particle) has a largest linear dimension (LLD), e.g., mean diameter, or size that is at least about 0.5 millimeter (mm), preferably about 1.0 mm, about 1.5 mm or greater. In some embodiments, a device can be as large as 10 mm in diameter or size. For example, a device or particle described herein is in a size range of 0.5 mm to 10 mm, 1 mm to 10 mm, 1 mm to 8 mm, 1 mm to 6 mm, 1 mm to 5 mm, 1 mm to 4 mm, 1 mm to 3 mm, 1 mm to 2 mm, 1 mm to 1.5 mm, 1.5 mm to 8 mm, 1.5 mm to 6 mm, 1.5 mm to 5 mm, 1.5 mm to 4 mm, 1.5 mm to 3 mm, 1.5 mm to 2 mm, 2 mm to 8 mm, 2 mm to 7 mm, 2 mm to 6 mm, 2 mm to 5 mm, 2 mm to 4 mm, 2 mm to 3 mm, 2.5 mm to 8 mm, 2.5 mm to 7 mm, 2.5 mm to 6 mm, 2.5 mm to 5 mm, 2.5 mm to 4 mm, 2.5 mm to 3 mm, 3 mm to 8 mm, 3 mm to 7 mm, 3 mm to 6 mm, 3 mm to 5 mm, 3 mm to 4 mm, 3.5 mm to 8 mm, 3.5 mm to 7 mm, 3.5 mm to 6 mm, 3.5 mm to 5 mm, 3.5 mm to 4 mm, 4 mm to 8 mm, 4 mm to 7 mm, 4 mm to 6 mm, 4 mm to 5 mm, 4.5 mm to 8 mm, 4.5 mm to 7 mm, 4.5 mm to 6 mm, 4.5 mm to 5 mm, 5 mm to 8 mm, 5 mm to 7 mm, 5 mm to 6 mm, 5.5 mm to 8 mm, 5.5 mm to 7 mm, 5.5 mm to 6 mm, 6 mm to 8 mm, 6 mm to 7 mm, 6.5 mm to 8 mm, 6.5 mm to 7 mm, 7 mm to 8 mm, or 7.5 mm to 8 mm.

In some embodiments, a device of the disclosure (e.g., particle, capsule) comprises at least one pore or opening, e.g., to allow for the free flow of materials. In some embodiments, the mean pore size of a device is between about 0.1 μm to about 10 μm. For example, the mean pore size may be between 0.1 μm to 10 μm, 0.1 μm to 5 μm, 0.1 μm to 2 μm, 0.15 μm to 10 μm, 0.15 μm to 5 μm, 0.15 μm to 2 μm, 0.2 μm to 10 μm, 0.2 μm to 5 μm, 0.25 μm to 10 μm, 0.25 μm to 5 μm, 0.5 μm to 10 μm, 0.75 μm to 10 μm, 1 μm to 10 μm, 1 μm to 5 μm, 1 μm to 2 μm, 2 μm to 10 μm, 2 μm to 5 μm, or 5 μm to 10 μm. In some embodiments, the mean pore size of a device is between about 0.1 μm to 10 μm. In some embodiments, the mean pore size of a device is between about 0.1 μm to 5 μm. In some embodiments, the mean pore size of a device is between about 0.1 μm to 1 μm.

In some embodiments, the device comprises a semi-permeable, biocompatible membrane surrounding the genetically modified cells that are encapsulated in a polymer composition (e.g., an alginate hydrogel). The membrane pore size is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

In an embodiment, the device may contain a cell-containing compartment that is surrounded with a barrier compartment formed from a cell-free biocompatible material, such as the core-shell microcapsules described in Ma, M et al., Adv. Healthc Mater., 2(5):667-672 (2012). Such a barrier compartment could be used with or without the semi-permeable membrane.

Cells in the cell-containing compartment(s) of a device of the disclosure may be encapsulated in a polymer composition. The polymer composition may comprise one or more hydrogel-forming polymers. In addition to the polymer composition in the cell-containing compartment(s), the device (e.g., macrodevice, particle, hydrogel capsule) may comprise or be formed from materials such as metals, metallic alloys, ceramics, polymers, fibers, inert materials, and combinations thereof. A device may be completely made up of one type of material, or may comprise other materials within the cell-containing compartment and any other compartments.

In some embodiments, the device comprises a metal or a metallic alloy. In an embodiment, one or more of the compartments in the device (e.g., the first compartment, the second compartment, or all compartments) comprises a metal or a metallic alloy. Exemplary metallic or metallic alloys include comprising titanium and titanium group alloys (e.g., nitinol, nickel titanium alloys, thermo-memory alloy materials), platinum, platinum group alloys, stainless steel, tantalum, palladium, zirconium, niobium, molybdenum, nickel-chrome, chromium molybdenum alloys, or certain cobalt alloys (e.g., cobalt-chromium and cobalt-chromium-nickel alloys, e.g., ELGILOY® and PHYNOX®). For example, a metallic material may be stainless steel grade 316 (SS 316L) (comprised of Fe, <0.3% C, 16-18.5% Cr, 10-14% Ni, 2-3% Mo, <2% Mn, <1% Si, <0.45% P, and <0.03% S). In metal-containing devices, the amount of metal (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device comprises a ceramic. In an embodiment, one or more of the compartments in the device (e.g., the first compartment, the second compartment, or all compartments) comprises a ceramic. Exemplary ceramic materials include oxides, carbides, or nitrides of the transition elements, such as titanium oxides, hafnium oxides, iridium oxides, chromium oxides, aluminum oxides, and zirconium oxides. Silicon based materials, such as silica, may also be used. In ceramic-containing devices, the amount of ceramic (e.g., by % weight, actual weight) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment. In such embodiments, the thickness of the second (outer) compartment means the average distance between the outer boundary of the second compartment and the interface between the two compartments, e.g., the average of the distances measured at each of the thinnest and thickest points visually observed in the outer compartment. In some embodiments (e.g., the device is about 1.5 mm in diameter), the thinnest and thickest distances for the outer compartment are between 25 and 110 micrometers (μm) and between 270 and 480 μm, respectively. In some embodiments, the thickness of the outer compartment is greater than about 10 nanometers (nm), preferably 100 nm or greater and can be as large as 1 millimeter (mm). For example, the thickness (e.g., average distance) of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness (e.g., average distance) of the outer compartment is 100 nm to 1 mm, between 1 μm and 1 mm, between 1 μm and 500 μm or between 5 μm and 1 mm. In some embodiments, the thickness (e.g., average distance) of the outer compartment is between about 50 μm and about 100 μm. In some embodiments (e.g., the device is about 1.5 mm in diameter), the thickness of the outer compartment (e.g., average distance) is between about 180 μm and 260 μm or between about 310 μm and 440 μm.

In some embodiments of a two-compartment hydrogel capsule device, the mean pore size of the cell-containing inner compartment and the outer compartment is substantially the same. In some embodiments, the mean pore size of the inner compartment and the second compartment differ by about 1.5%, 2%, 5%, 7.5%, 10%, 15%, 20%, 25%, 30%, 35%, 40%, 45%, 50%, 55%, 60%, 65%, 70%, 75%, or more. In some embodiments, the mean pore size of the device (e.g., mean pore size of the first compartment and/or mean pore size of the second compartment) is dependent on a number of factors, such as the material(s) within each compartment and the presence and density of a compound of Formula (I).

In some embodiments, the polymer composition in the cell-containing compartment(s) comprises a polysaccharide or other hydrogel-forming polymer (e.g., alginate, hyaluronate or chondroitin). In some embodiments, the polymer is an alginate, which is a polysaccharide made up of β-D-mannuronic acid (M) and α-L-guluronic acid (G). In some embodiments, the alginate has a low molecular weight (e.g., approximate molecular weight of <75 kD) and G:M ratio ≥1.5, (ii) a medium molecular weight alginate, e.g., has approximate molecular weight of 75-150 kDa and G:M ratio ≥1.5, (iii) a high molecular weight algiinate, e.g., has an approximate MW of 150 kDa-250 kDa and G:M ratio ≥0.5, (iv) or a blend of two or more of these alginates. In some embodiments, the cell-containing compartment(s) further comprises at least one cell-binding substance (CBS), e.g., a cell-binding peptide (CBP) or cell-binding polypeptide (CBPP) described in WO2020069429.

In some embodiments, the cell-containing compartment(s) comprises an alginate covalently modified with a linker-cell-binding peptide moiety, e.g., GRGD (SEQ ID NO:57) or GRGDSP (SEQ ID NO:58). In an embodiment, the cell-binding peptide density in the cell-containing compartment(s) (% nitrogen as determined by combustion analysis, e.g., as described in WO2020198695) to be at least 0.05%, 0.1%, 0.2% or 0.3% but less than 4%, 3%, 2% or 1%. In an embodiment, the total density of the linker-CBP in a cell containing compartment is about 0.1 to about 1.0 micromoles of the CBP per g of CBP-polymer (e.g., a MMW-alginate covalently modified with GRGD (SEQ ID NO:57) or GRGDSP (SEQ ID NO:58) in solution as determined by a quantitative peptide conjugation assay, e.g., an assay described in WO2020198695. In an embodiment, the linker-CBP is GRGDSP and the alginate has a molecular weight of 75 kDa to 150 kDa and a G:M ratio of greater than or equal to 1.5. In an embodiment, the cell-containing compartment also comprises an unmodified alginate with a molecular weight of 75 kDa to 150 kDa and a G:M ratio of greater than or equal to 1.5.

The device may form part of a plurality of substantially the same devices in a preparation (e.g., composition). In some embodiments, the devices (e.g., particles, hydrogel capsules) in the preparation have a mean diameter or size between about 0.5 mm to about 8 mm. In some embodiments, the mean diameter or size of devices in the preparation is between about 0.5 mm to about 4 mm or between about 0.5 mm to about 2 mm. In some embodiments, the devices in the preparation are two-compartment hydrogel capsules and have a mean diameter or size of about 0.7 mm to about 1.3 mm or about 1.2 mm to about 1.8 mm.

In some embodiments, the surface of the device comprises a compound capable of mitigating the FBR upon implant into a subject, an afibrotic compound as described herein below. For devices comprising a barrier compartment surrounding the cell-containing compartment, the afibrotic compound may covalently modify a polymer disposed throughout the barrier compartment and optionally throughout the cell-containing compartment.

In some embodiments, one or more compartments in a device comprises an afibrotic polymer, e.g., an afibrotic compound of Formula (I) covalently attached to a polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a 2-compartment hydrogel capsule, the afibrotic polymer is present only in the outer, barrier compartment.

One or more compartments in a device may comprise an unmodified polymer that is the same or different than the polymer in any afibrotic polymer that is present in the device. In an embodiment, the first compartment, second compartment or all compartments in the device comprise the unmodified polymer.

Each of the modified and unmodified polymers in the device may be a linear, branched, or cross-linked polymer, or a polymer of selected molecular weight ranges, degree of polymerization, viscosity or melt flow rate. Branched polymers can include one or more of the following types: star polymers, comb polymers, brush polymers, dendronized polymers, ladders, and dendrimers. A polymer may be a thermoresponsive polymer, e.g., gel (e.g., becomes a solid or liquid upon exposure to heat or a certain temperature) or a photocrosslinkable polymer. Exemplary polymers include polystyrene, polyethylene, polypropylene, polyacetylene, poly(vinyl chloride) (PVC), polyolefin copolymers, poly(urethane)s, polyacrylates and polymethacrylates, polyacrylamides and polymethacrylamides, poly(methyl methacrylate), poly(2-hydroxyethyl methacrylate), polyesters, polysiloxanes, polydimethylsiloxane (PDMS), polyethers, poly(orthoester), poly(carbonates), poly(hydroxyalkanoate)s, polyfluorocarbons, PEEK®, Teflon® (polytetrafluoroethylene, PTFE), PEEK, silicones, epoxy resins, Kevlar®, Dacron® (a condensation polymer obtained from ethylene glycol and terephthalic acid), polyethylene glycol, nylon, polyalkenes, phenolic resins, natural and synthetic elastomers, adhesives and sealants, polyolefins, polysulfones, polyacrylonitrile, biopolymers such as polysaccharides and natural latex, collagen, cellulosic polymers (e.g., alkyl celluloses, etc.), polyethylene glycol and 2-hydroxyethyl methacrylate (HEMA), polysaccharides, poly(glycolic acid), poly(L-lactic acid) (PLLA), poly(lactic glycolic acid) (PLGA), a polydioxanone (PDA), or racemic poly(lactic acid), polycarbonates, (e.g., polyamides (e.g., nylon)), fluoroplastics, carbon fiber, agarose, alginate, chitosan, and blends or copolymers thereof. In polymer-containing devices, the amount of a polymer (e.g., by % weight of the device, actual weight of the polymer) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

In some embodiments, one or more of the modified and unmodified polymers in the device comprises a polyethylene. Exemplary polyethylenes include ultra-low-density polyethylene (ULDPE) (e.g., with polymers with densities ranging from 0.890 to 0.905 g/cm³, containing comonomer); very-low-density polyethylene (VLDPE) (e.g., with polymers with densities ranging from 0.905 to 0.915 g/cm³, containing comonomer); linear low-density polyethylene (LLDPE) (e.g., with polymers with densities ranging from 0.915 to 0.935 g/cm³, contains comonomer); low-density polyethylene (LDPE) (e.g., with polymers with densities ranging from about 0.915 to 0.935 g/m³); medium density polyethylene (MDPE) (e.g., with polymers with densities ranging from 0.926 to 0.940 g/cm³, may or may not contain comonomer); high-density polyethylene (HDPE) (e.g., with polymers with densities ranging from 0.940 to 0.970 g/cm³, may or may not contain comonomer) and polyethylene glycol.

In some embodiments, one or more of the modified and unmodified polymers in the device comprises a polypropylene. Exemplary polypropylenes include homopolymers, random copolymers (homophasic copolymers), and impact copolymers (heterophasic copolymers), e.g., as described in McKeen, Handbook of Polymer Applications in Medicine and Medical Devices, 3-Plastics Used in Medical Devices, (2014):21-53.

In some embodiments, one or more of the modified and unmodified polymers in the device comprises a polypropylene. Exemplary polystyrenes include general purpose or crystal (PS or GPPS), high impact (HIPS), and syndiotactic (SPS) polystyrene.

In some embodiments, one or more of the modified and unmodified polymers comprises a comprises a thermoplastic elastomer (TPE). Exemplary TPEs include (i) TPA-polyamide TPE, comprising a block copolymer of alternating hard and soft segments with amide chemical linkages in the hard blocks and ether and/or ester linkages in the soft blocks; (ii) TPC-co-polyester TPE, consisting of a block copolymer of alternating hard segments and soft segments, the chemical linkages in the main chain being ester and/or ether; (iii) TPO-olefinic TPE, consisting of a blend of a polyolefin and a conventional rubber, the rubber phase in the blend having little or no cross-linking; (iv) TPS-styrenic TPE, consisting of at least a triblock copolymer of styrene and a specific diene, where the two end blocks (hard blocks) are polystyrene and the internal block (soft block or blocks) is a polydiene or hydrogenated polydiene; (v) TPU-urethane TPE, consisting of a block copolymer of alternating hard and soft segments with urethane chemical linkages in the hard blocks and ether, ester or carbonate linkages or mixtures of them in the soft blocks; (vi) TPV-thermoplastic rubber vulcanizate consisting of a blend of a thermoplastic material and a conventional rubber in which the rubber has been cross-linked by the process of dynamic vulcanization during the blending and mixing step; and (vii) TPZ-unclassified TPE comprising any composition or structure other than those grouped in TPA, TPC, TPO, TPS, TPU, and TPV.

In some embodiments, the unmodified polymer is an unmodified alginate. In some embodiments, the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In an embodiment, the unmodified alginate has a molecular weight of 150 kDa-250 kDa and a G:M ratio of ≥1.5.

In some embodiments, the afibrotic polymer comprises an alginate chemically modified with a Compound of Formula (1) The alginate in the afibrotic polymer may be the same or different than any unmodified alginate that is present in the device. In an embodiment, the density of the Compound of Formula (I) in the afibrotic alginate (e.g., amount of conjugation) is between about 4.0% and about 8.0%, between about 5.0% and about 7.0%, or between about 6.0% and about 7.0% nitrogen (e.g., as determined by combustion analysis for percent nitrogen). In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described in WO2020069429), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w.

In alginate-containing devices, the amount of modified and unmodified alginates (e.g., by % weight of the device, actual weight of the alginate) can be at least 5%, e.g., at least 5%, 10%, 20%, 30%, 40%, 50%, 60%, 70%, 80%, 90%, 95%, 99%, or more, e.g., w/w; less than 20%, e.g., less than 20%, 15%, 10%, 5%, 1%, 0.5%, 0.1%, or less.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with an activating agent (e.g., 2-chloro-4,6-dimethoxy-1,3,5-triazine (0.5 eq)) and a base (e.g., N-methylmorpholine (1 eq)). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate. The alginate in an afibrotic polymer can be chemically modified with a compound of Formula (I) using any suitable method known in the art, e.g., as described in any of WO 2021/119522, WO 2019/195055, WO 2018/067615, WO 2017/075631, WO 2016/019391 and WO 2012/167223.

In an embodiment, the device comprises at least one cell-containing compartment, and in some embodiments contains two, three, four or more cell-containing compartments. In an embodiment, each cell-containing compartment comprises a plurality of cells (e.g., live cells) and the cells in at least one of the compartments are capable of expressing and secreting an ARSB fusion protein when the device is implanted into a subject.

In an embodiment, all the cells in a cell-containing compartment are derived from a single parental cell-type or a mixture of at least two different parental cell types. In an embodiment, all of the cells in a cell-containing compartment are derived from the same parental cell type, but a first plurality of the derived cells are engineered to express the ARSB fusion protein, and a second plurality of the derived cells are engineered to express a different therapeutic protein. In devices with two or more cell-containing compartments, the cells and the protein(s) produced thereby may be the same or different in each cell-containing compartment. In some embodiments, all of the cell-containing compartments are surrounded by a single barrier compartment. In some embodiments, the barrier compartment is substantially cell-free.

In an embodiment, cells to be incorporated into a device described herein, e.g., a hydrogel capsule, are prepared in the form of a cell suspension prior to being encapsulated within the device. The cells in the suspension may take the form of single cells (e.g., from a monolayer cell culture), or provided in another form, e.g., disposed on a microcarrier (e.g., a bead or matrix) or as a three-dimensional aggregate of cells (e.g., a cell cluster or spheroid). The cell suspension can comprise multiple cell clusters (e.g., as spheroids) or microcarriers.

In addition to the fusion protein secreted by the encapsulated cells, a device (e.g., capsule, particle) may comprise one or more exogenous agents that are not expressed by the cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), an active or inactive fragment of a protein or polypeptide, a small molecule, or drug. In an embodiment, the device is configured to release such an exogenous agent.

### Afibrotic (e.g., FBR-Mitigating) Compounds

In some embodiments, the devices described herein comprise at least one compound of Formula (I):

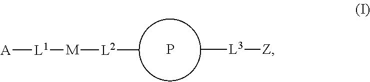

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocyclyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, —N(R^{C})(R^{D}), cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{Cl})(R^{D1}), —P(R^{F1}), cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the compound of Formula (I) is a compound of Formula (I-a):

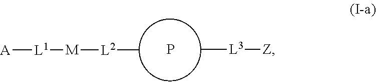

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D}), N(R^{C})C(O)(C₁-C₆-alkylene)-, —N(R^{C})C(O)(C₁-C₆-alkenylene)-, —NCN—, —C(═N(R^{C})(R^{D}))O—, —S—, —S(O)ₓ, —OS(O)ₓ, —N(R^{C})S(O)ₓ, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is heteroaryl optionally substituted by one or more R⁴;

Z is alkyl, alkenyl, alkynyl, heteroalkyl, cycloalkyl, heterocyclyl, aryl, or heteroaryl, each of which is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, for Formulas (I) and (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{C})—, and R^{c} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) and (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. I n some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) and (I-a), L³ is a bond, alkyl, or heteroalkyl. In some embodiments, L³ is a bond. In some embodiments, L³ is alkyl. In some embodiments, L³ is C₁-C₁₂ alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) and (I-a), M is absent, alkyl, heteroalkyl, aryl, or heteroaryl. In some embodiments, for Formulas (I) and (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂—)_{z}, wherein z is an integer selected from 1 to 10. In some embodiments, z is an integer selected from 1 to 5. In some embodiments, M is —(OCH₂)₂—, (—OCH₂CH₂—)₂, (—OCH₂CH₂—)₃, (—OCH₂CH₂—)₄, or (—OCH₂CH_{2 5}. In some embodiments, M is —OCH₂CH₂—, (—OCH₂CH₂₋₎₂, (—OCH₂CH₂—)₃, or (—OCH₂CH₂₋₎₄. In some embodiments, M is (—OCH₂—)₃. In some embodiments, M is aryl. In some embodiments, M is phenyl. In some embodiments, M is unsubstituted phenyl. In some embodiments, M is

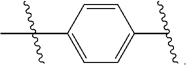 [SMILES: *c1ccc(*)cc1]

In some embodiments, M is

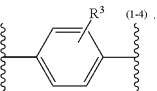

In some embodiments, M is phenyl substituted with 1-4 R³ (e.g., 1 R³). In some embodiments, R³ is CF₃.

In some embodiments, for Formulas (I) and (I-a), P is absent, heterocyclyl, or heteroaryl. In some embodiments, for Formulas (I) and (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, or pyrrolyl. In some embodiments, P is imidazolyl. In some embodiments, P is 1,2,3-triazolyl. In some embodiments, P is

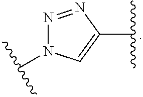 [SMILES: *c1cn(*)nn1]

In some embodiments, P is

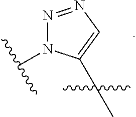 [SMILES: *c1cnnn1*]

In some embodiments, P is

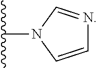 [SMILES: *n1ccnc1]

In some embodiments, P is heterocyclyl. In some embodiments, P is heterocyclyl. In some embodiments, P is a 5-membered heterocyclyl. In some embodiments, P is imidazolidinonyl. In some embodiments, P is

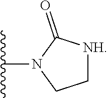 [SMILES: *N1CCNC1=O]

In some embodiments, P is thiomorpholinyl-1,1-dioxidyl. In some embodiments, P is

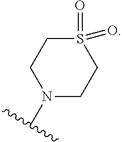 [SMILES: *N1CCS(=O)(=O)CC1]

In some embodiments, for Formulas (I) and (I-a), Z is alkyl, heteroalkyl, cycloalkyl, heterocyclyl, aryl, or heteroaryl. In some embodiments, for Formulas (I) and (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl, 5-membered heterocyclyl, or 6-membered heterocyclyl. In some embodiments, Z is a 6-membered oxygen-containing heterocyclyl. In some embodiments, Z is tetrahydropyranyl. In some embodiments, Z is

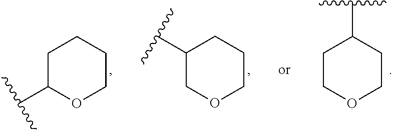 [SMILES: *C1CCCCO1 ; *C1CCCOC1 ; *C1CCOCC1]

In some embodiments, Z is a 4-membered oxygen-containing heterocyclyl. In some embodiments, Z is

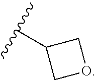 [SMILES: *C1COC1]

In some embodiments, Z is a bicyclic oxygen-containing heterocyclyl. In some embodiments, Z is a bicyclic oxygen-containing heterocyclyl. In some embodiments, Z is phthalic anhydridyl. In some embodiments, Z is a sulfur-containing heterocyclyl

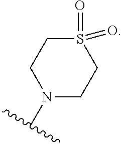 [SMILES: *N1CCS(=O)(=O)CC1]

In some embodiments, Z is a 6-membered sulfur-containing heterocyclyl

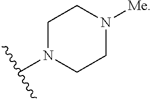 [SMILES: *N1CCN(C)CC1]

In some embodiments, Z is a 6-membered heterocyclyl containing a nitrogen atom and a sulfur atom. In some embodiments, Z is thiomorpholinyl-1,1-dioxidyl. In some embodiments, Z is

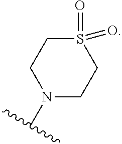 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

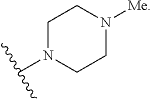 [SMILES: *N1CCN(C)CC1]

In some embodiments, Z is a bicyclic heterocyclyl. In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

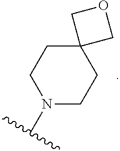 [SMILES: *N1CCC2(CC1)COC2]

In some embodiments, Z is 1-oxa-3,8-diazaspiro[4.5]decan-2-one. In some embodiments, Z is

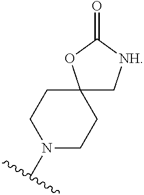 [SMILES: *N1CCC2(CC1)CNC(=O)O2]

In some embodiments, for Formulas (I) and (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) and (I-a), Z is alkyl. In some embodiments, Z is C₁-C₁₂ alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) and (I-a), Z is heteroalkyl. In some embodiments, Z is C₁-C₁₂ heteroalkyl. I n some embodiments, Z is C₁-C₁₀ heteroalkyl. In some embodiments, Z is C₁-C₈ heteroalkyl. I n some embodiments, Z is C₁-C₆ heteroalkyl. In some embodiments, Z is a nitrogen-containing heteroalkyl optionally substituted with one or more R⁵. I n some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

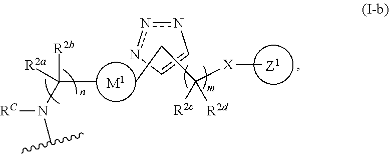

or a pharmaceutically acceptable salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R^{5′} and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

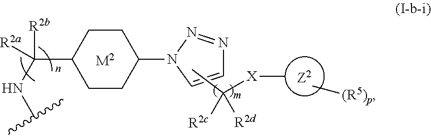

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii).

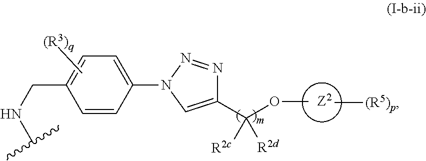

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

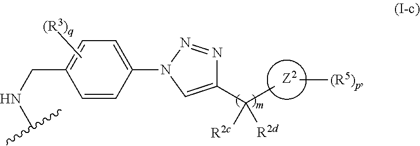

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR, —C(O)OR, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

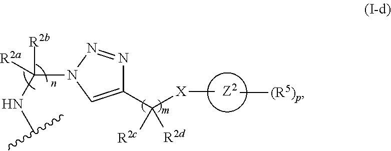

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

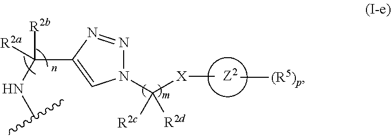

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a [symbol] polymer described herein.

In some embodiments, the compound of Formula (I) is a compound of Formula (I-f):

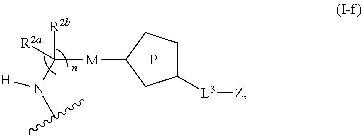

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

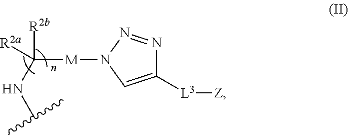

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR, wherein alkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

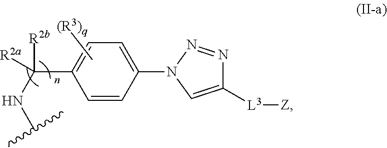

or a pharmaceutically acceptable salt thereof, wherein L³ is alkyl or heteroalkyl, each of which is optionally substituted with one or more R²; Z is hydrogen, alkyl, heteroalkyl, or —OR^{A}, heteroalkyl are optionally substituted with one or more R⁵; each of R^{2a} and R^{2b} is independently hydrogen, alkyl, or heteroalkyl, or each of R^{2a} and R^{2b} is taken together to form an oxo group, each R², R³, and R⁵ is independently heteroalkyl, halogen, oxo, —OR^{A1}, —C(O)OR^{A1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

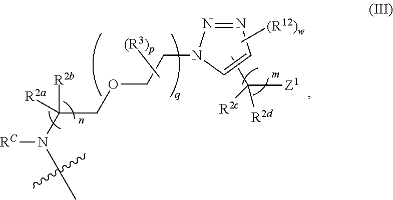

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or or each of R^{2a} and R^{2b} or R^{2c} and R^{2d} is taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; q is an integer from 0 to 25; w is 0 or 1; and “ [symbol] ” refers to a connection to an attachment group or a polymer described herein.

In some embodiments, the compound of Formula (III) is a compound of Formula (III-a):

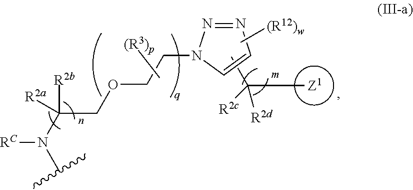

or a pharmaceutically acceptable salt thereof, wherein Ring Z¹ is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R¹² is independently deuterium, alkyl, heteroalkyl, haloalkyl, halo, cyano, nitro, or amino; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; w is 0 or 1; and “ [symbol] ” refers to a connection to an attachment group or a polymer described herein.

In some embodiments, the compound of Formula (III) is a compound of Formula (III-b):

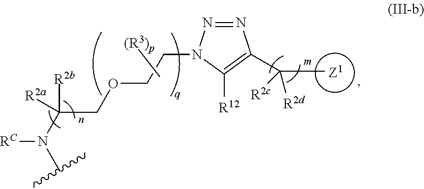

or a pharmaceutically acceptable salt thereof, wherein Ring Z¹ is cycloalkyl, heterocyclyl, aryl, or heteroaryl, each of which is optionally substituted with 1-5 R⁵; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; and “ [symbol] ” refers to a connection to an attachment group or a polymer described herein.

In some embodiments, the compound of Formula (III) is a compound of Formula (III-c):

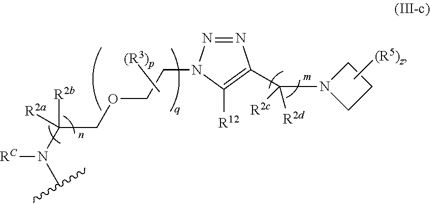

or a pharmaceutically acceptable salt thereof, wherein R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, the compound of Formula (III) is a compound of Formula (III-d):

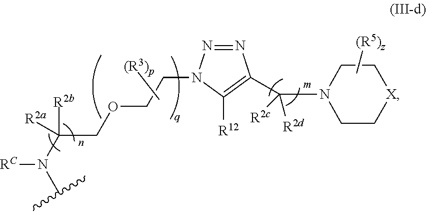

or a pharmaceutically acceptable salt thereof, wherein X is C(R′)(R″), N(R′), or S(O)ₓ; each of R′ and R″ is independently hydrogen, alkyl, or halogen; R^{C} is hydrogen, alkyl, —N(R^{C})C(O)R^{B}, —N(R^{C})C(O)(C₁-C₆-alkyl), or —N(R^{C})C(O)(C₁-C₆-alkenyl), wherein each of alkyl and alkenyl is optionally substituted with 1-6 R⁶; each of R^{2a}, R^{2b}, R^{2c}, and R^{2d} is independently hydrogen or alkyl; or R^{2a} and R^{2b} or R^{2c} and R^{2d} are taken together to form an oxo group; each of R³, R⁵, and R⁶ is independently alkyl, heteroalkyl, halogen, oxo, —OR^{A1}, —C(O)OR^{A1}, or —C(O)R^{B1}; R¹² is hydrogen, deuterium, alkyl, heteroalkyl, haloalkyl, halo, cyano, nitro, or amino; each R^{A1}, R^{B1} and R^{E1} is independently hydrogen, alkyl, or heteroalkyl; m and n are each independently 1, 2, 3, 4, 5, or 6; q is an integer from 0 to 25; x is 0, 1, or 2; z is 0, 1, 2, 3, 4, 5, or 6, and “ [symbol] ” refers to a connection to an attachment group or a polymer described herein.

In some embodiments, X is S(O)ₓ. In some embodiments, x is 2. In some embodiments, X is S(O)₂.

In some embodiments, each of R^{2a}, R^{2b}, R^{2c}, and R^{2d} is independently hydrogen.

In some embodiments, R^{C} is hydrogen, —C(O)(C₁-C₆-alkyl), or —C(O)(C₁-C₆-alkenyl). In some embodiments, each of alkyl and alkenyl is substituted with 1 R⁶ (e.g., —CH₃). In some embodiments, R^{C} is hydrogen.

In some embodiments, n is 1. In some embodiments, q is 2, 3, 4, or 5. In some embodiments, q is 3. In some embodiments, m is 1. In some embodiments, p is 0. In some embodiments, R¹² is halo (e.g., Cl).

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Zⁱ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., a nitrogen-containing heterocyclyl, e.g., a nitrogen-containing spiro heterocyclyl, e.g., 2-oxa-7-azaspiro[3.5]nonanyl). In some embodiments, the compound of Formula (III-b) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 5 herein below, or a pharmaceutically acceptable salt thereof. In some embodiments, the exterior surface and/or one or more compartments within a device described herein comprises a small molecule compound shown in Table 5, or a pharmaceutically acceptable salt thereof.

TABLE 5
Exemplary afibrotic (FBR-mitigating) compounds

| Compound No. | Structure |
|---|---|
| 100 | 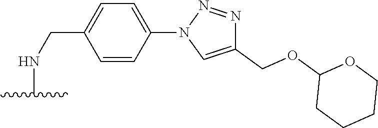 [SMILES: *NCc1ccc(-n2cc(COC3CCCCO3)nn2)cc1] |
| 101 | 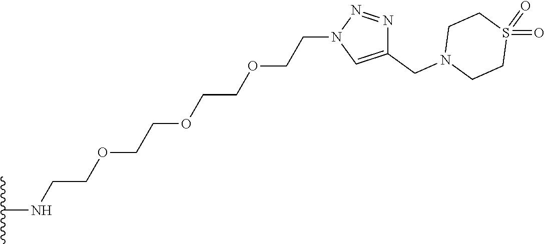 [SMILES: *NCCOCCOCCOCCn1cc(CN2CCS(=O)(=O)CC2)nn1] |
| 102 | 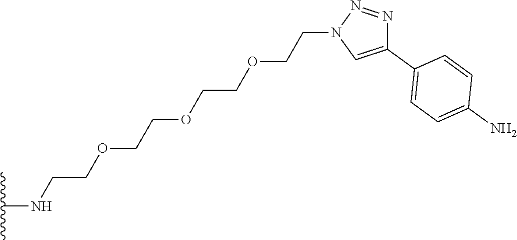 [SMILES: *NCCOCCOCCOCCn1cc(-c2ccc(N)cc2)nn1] |
| 103 | 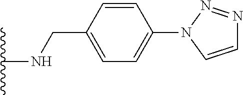 [SMILES: *NCc1ccc(-n2ccnn2)cc1] |
| 104 | 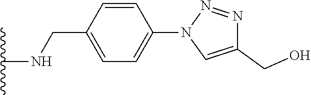 [SMILES: *NCc1ccc(-n2cc(CO)nn2)cc1] |
| 105 | 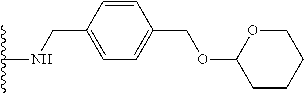 [SMILES: *NCc1ccc(COC2CCCCO2)cc1] |
| 106 | 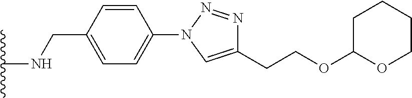 [SMILES: *NCc1ccc(-n2cc(CCOC3CCCCO3)nn2)cc1] |
| 107 | 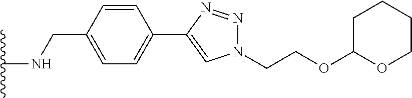 [SMILES: *NCc1ccc(-c2cn(CCOC3CCCCO3)nn2)cc1] |
| 108 | 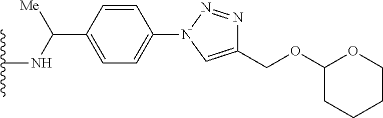 [SMILES: *NC(C)c1ccc(-n2cc(COC3CCCCO3)nn2)cc1] |
| 109 | 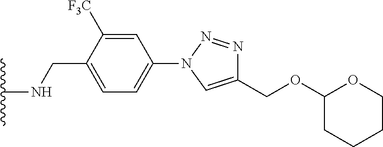 [SMILES: *NCc1ccc(-n2cc(COC3CCCCO3)nn2)cc1C(F)(F)F] |
| 110 | 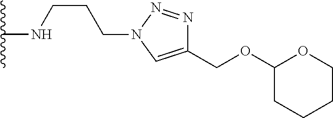 [SMILES: *NCCCn1cc(COC2CCCCO2)nn1] |
| 111 | 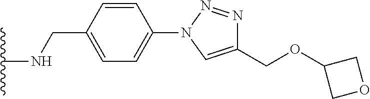 [SMILES: *NCc1ccc(-n2cc(COC3COC3)nn2)cc1] |
| 112 | 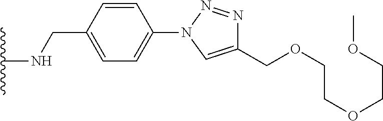 [SMILES: *NCc1ccc(-n2cc(COCCOCCOC)nn2)cc1] |
| 113 | 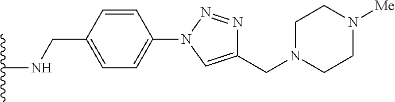 [SMILES: *NCc1ccc(-n2cc(CN3CCN(C)CC3)nn2)cc1] |
| 114 | 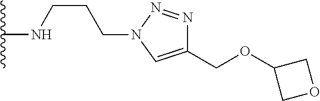 [SMILES: *NCCCn1cc(COC2COC2)nn1] |
| 115 | 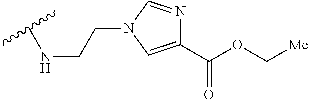 [SMILES: *NCCn1cnc(C(=O)OCC)c1] |
| 116 | 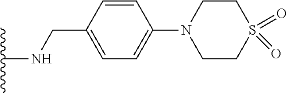 [SMILES: *NCc1ccc(N2CCS(=O)(=O)CC2)cc1] |
| 117 | 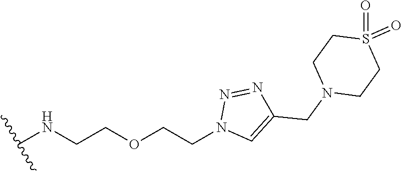 [SMILES: *NCCOCCn1cc(CN2CCS(=O)(=O)CC2)nn1] |
| 118 | 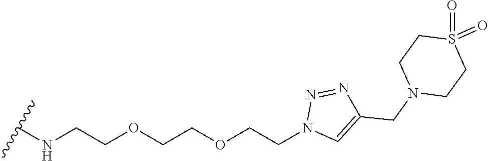 [SMILES: *NCCOCCOCCn1cc(CN2CCS(=O)(=O)CC2)nn1] |
| 119 | 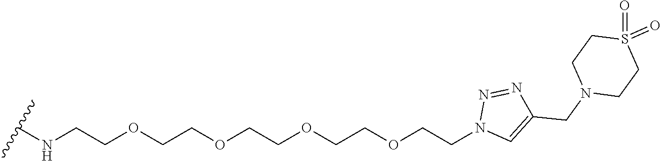 [SMILES: *NCCOCCOCCOCCOCCn1cc(CN2CCS(=O)(=O)CC2)nn1] |
| 120 | 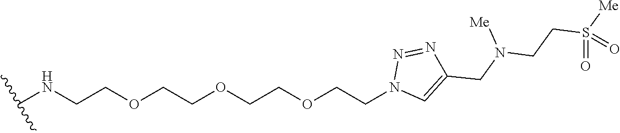 [SMILES: *NCCOCCOCCOCCn1cc(CN(C)CCS(C)(=O)=O)nn1] |
| 121 | 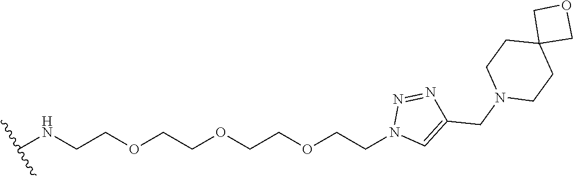 [SMILES: *NCCOCCOCCOCCn1cc(CN2CCC3(CC2)COC3)nn1] |
| 122 | 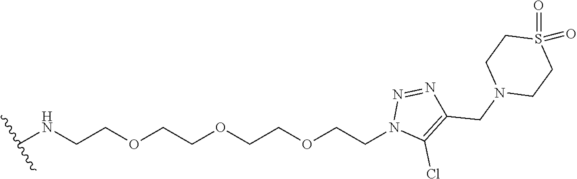 [SMILES: *NCCOCCOCCOCCn1nnc(CN2CCS(=O)(=O)CC2)c1Cl] |
| 123 | 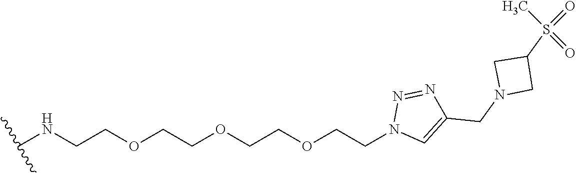 [SMILES: *NCCOCCOCCOCCn1cc(CN2CC(S(C)(=O)=O)C2)nn1] |
| 124 | 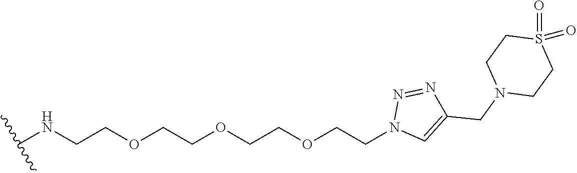 [SMILES: *NCCOCCOCCOCCn1cc(CN2CCS(=O)(=O)CC2)nn1] |
| 125 | 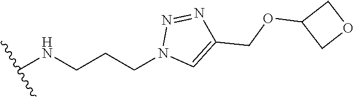 [SMILES: *NCCCn1cc(COC2COC2)nn1] |
| 126 | 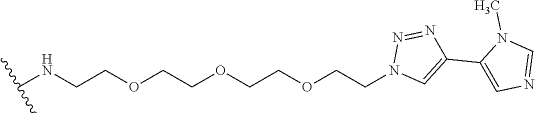 [SMILES: *NCCOCCOCCOCCn1cc(-c2cncn2C)nn1] |
| 127 | 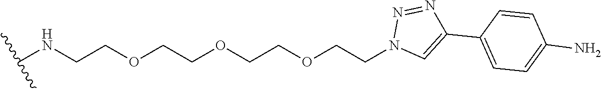 [SMILES: *NCCOCCOCCOCCn1cc(-c2ccc(N)cc2)nn1] |
| 128 | 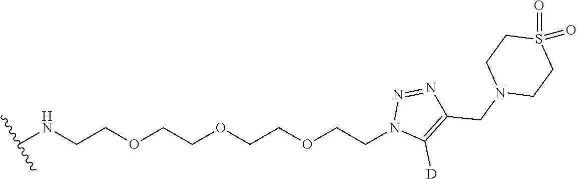 [SMILES: *NCCOCCOCCOCCn1nnc(CN2CCS(=O)(=O)CC2)c1[2H]] |
| 129 | 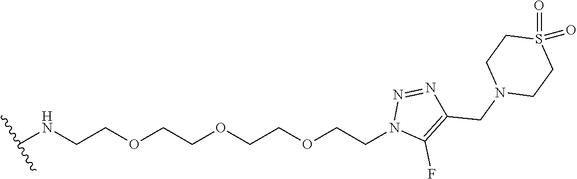 [SMILES: *NCCOCCOCCOCCn1nnc(CN2CCS(=O)(=O)CC2)c1F] |
| 130 | 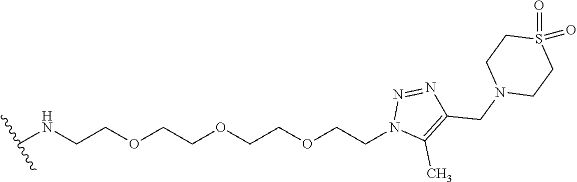 [SMILES: *NCCOCCOCCOCCn1nnc(CN2CCS(=O)(=O)CC2)c1C] |
| 131 | 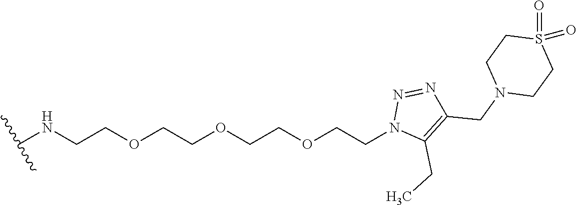 [SMILES: *NCCOCCOCCOCCn1nnc(CN2CCS(=O)(=O)CC2)c1CC] |
| 132 | 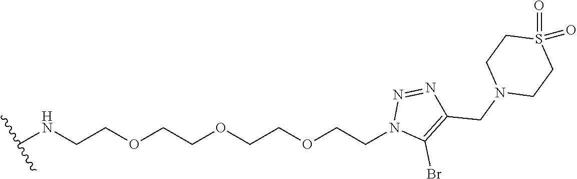 [SMILES: *NCCOCCOCCOCCn1nnc(CN2CCS(=O)(=O)CC2)c1Br] |
| 133 | 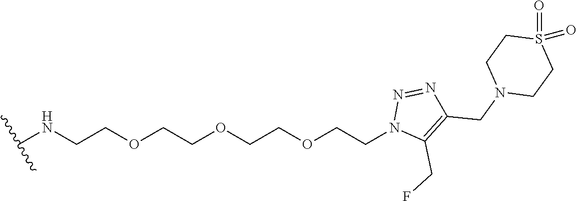 [SMILES: *NCCOCCOCCOCCn1nnc(CN2CCS(=O)(=O)CC2)c1CF] |
| 134 | 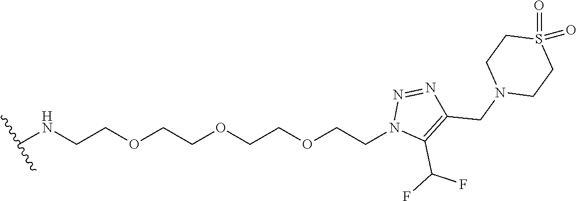 [SMILES: *NCCOCCOCCOCCn1nnc(CN2CCS(=O)(=O)CC2)c1C(F)F] |
| 135 | 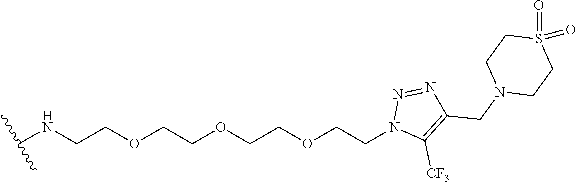 [SMILES: *NCCOCCOCCOCCn1nnc(CN2CCS(=O)(=O)CC2)c1C(F)(F)F] |
| 136 | 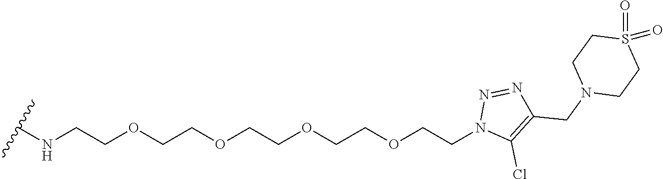 [SMILES: *NCCOCCOCCOCCOCCn1nnc(CN2CCS(=O)(=O)CC2)c1Cl] |
| 137 | 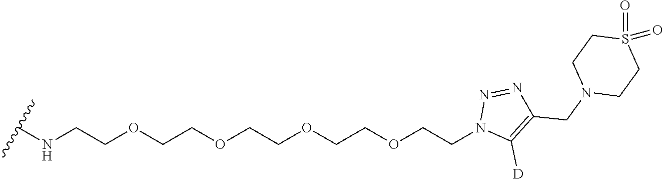 [SMILES: *NCCOCCOCCOCCOCCn1nnc(CN2CCS(=O)(=O)CC2)c1[2H]] |
| 138 | 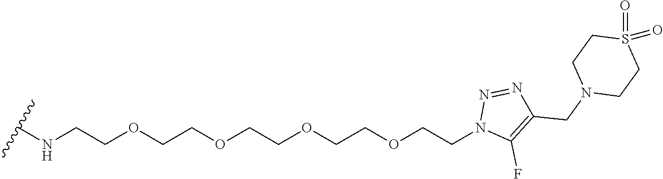 [SMILES: *NCCOCCOCCOCCOCCn1nnc(CN2CCS(=O)(=O)CC2)c1F] |
| 139 | 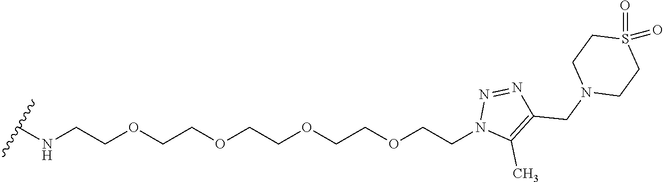 [SMILES: *NCCOCCOCCOCCOCCn1nnc(CN2CCS(=O)(=O)CC2)c1C] |
| 140 | 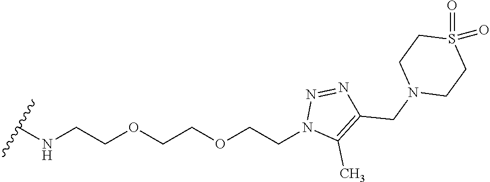 [SMILES: *NCCOCCOCCn1nnc(CN2CCS(=O)(=O)CC2)c1C] |
| 141 | 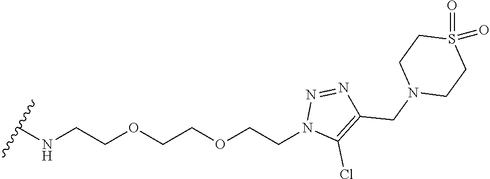 [SMILES: *NCCOCCOCCn1nnc(CN2CCS(=O)(=O)CC2)c1Cl] |
| 142 | 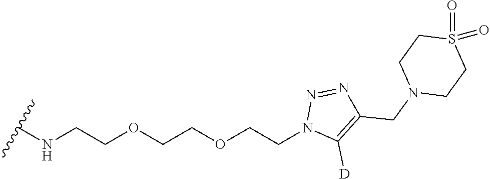 [SMILES: *NCCOCCOCCn1nnc(CN2CCS(=O)(=O)CC2)c1[2H]] |
| 143 | 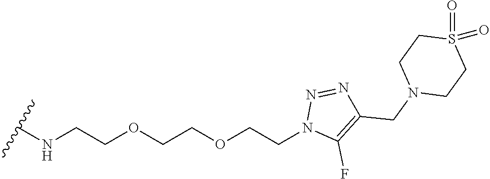 [SMILES: *NCCOCCOCCn1nnc(CN2CCS(=O)(=O)CC2)c1F] |
| 144 | 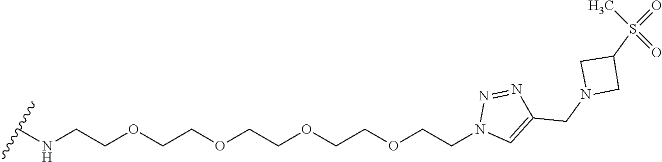 [SMILES: *NCCOCCOCCOCCOCCn1cc(CN2CC(S(C)(=O)=O)C2)nn1] |
| 145 | 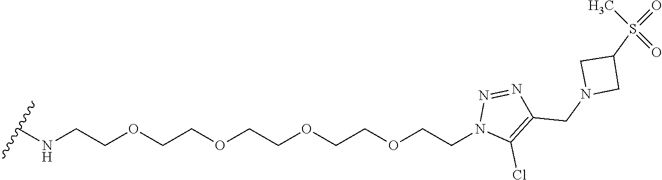 [SMILES: *NCCOCCOCCOCCOCCn1nnc(CN2CC(S(C)(=O)=O)C2)c1Cl] |
| 146 | 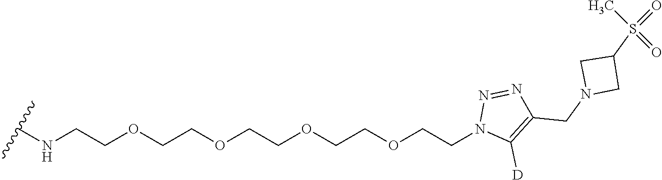 [SMILES: *NCCOCCOCCOCCOCCn1nnc(CN2CC(S(C)(=O)=O)C2)c1[2H]] |
| 147 | 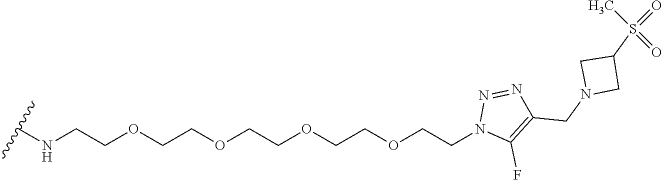 [SMILES: *NCCOCCOCCOCCOCCn1nnc(CN2CC(S(C)(=O)=O)C2)c1F] |
| 148 | 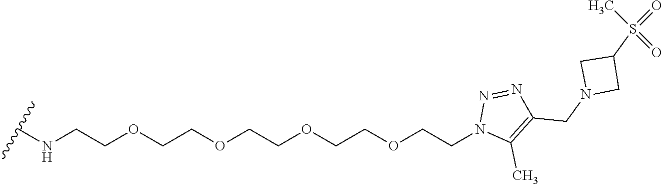 [SMILES: *NCCOCCOCCOCCOCCn1nnc(CN2CC(S(C)(=O)=O)C2)c1C] |
| 149 | 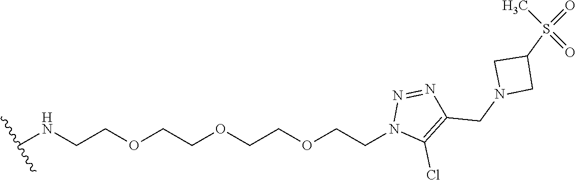 [SMILES: *NCCOCCOCCOCCn1nnc(CN2CC(S(C)(=O)=O)C2)c1Cl] |
| 150 | 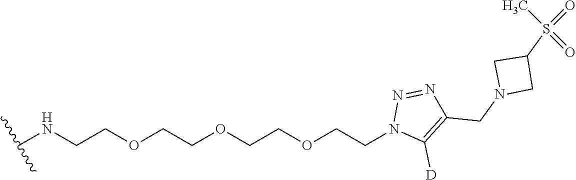 [SMILES: *NCCOCCOCCOCCn1nnc(CN2CC(S(C)(=O)=O)C2)c1[2H]] |
| 151 | 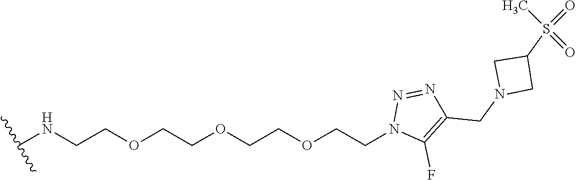 [SMILES: *NCCOCCOCCOCCn1nnc(CN2CC(S(C)(=O)=O)C2)c1F] |
| 152 | 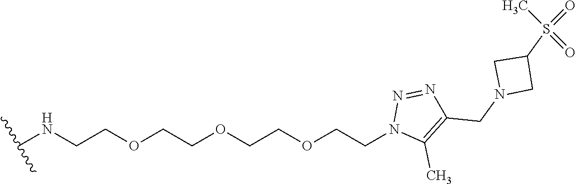 [SMILES: *NCCOCCOCCOCCn1nnc(CN2CC(S(C)(=O)=O)C2)c1C] |
| 153 | 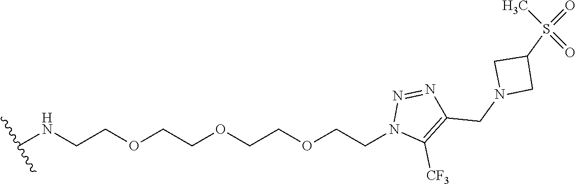 [SMILES: *NCCOCCOCCOCCn1nnc(CN2CC(S(C)(=O)=O)C2)c1C(F)(F)F] |
| 154 | 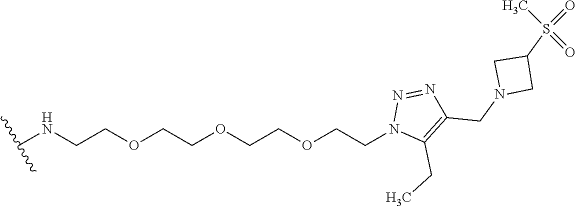 [SMILES: *NCCOCCOCCOCCn1nnc(CN2CC(S(C)(=O)=O)C2)c1CC] |

Conjugation of any of the compounds in Table 5 to a polymer (e.g., an alginate) may be performed as described in Example 2 of WO 2019/195055 or any other suitable chemical reaction.

In some embodiments, the compound is a compound of Formula (I) (e.g., Formulas (I-a), (I-b), (I-c), (I-d), (I-e), (I-f), (II), (II-a), (III), (III-a), (III-b), (III-c), or (III-d), or a pharmaceutically acceptable salt thereof and is selected from:

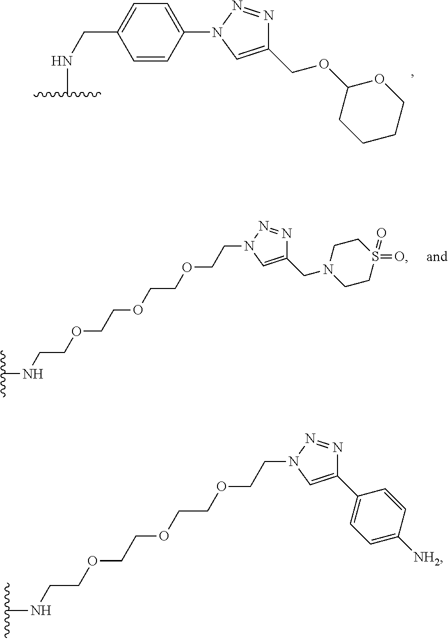 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt thereof.

In some embodiments, the device described herein comprises the compound of

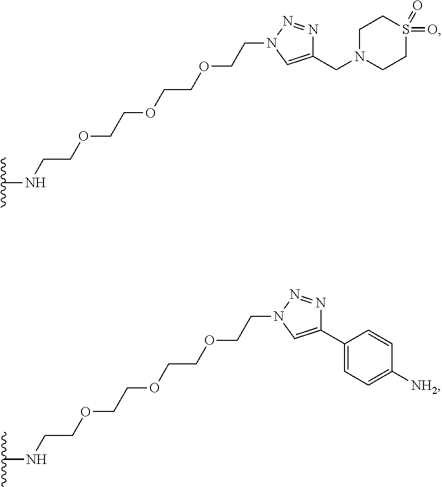 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.
In some embodiments, a compound of Formula (I) (e.g., Compound 101 in Table 5) is covalently attached to an alginate (e.g., an alginate with approximate MW<75 kDa, G:M ratio ≥1.5) at a conjugation density of at least 2.0% and less than 9.0%, or 3.0% to 8.0%, 4.0-7.0, 5.0 to 7.0, or 6.0 to 7.0 or about 6.8 as determined by combustion analysis for percent nitrogen as described in WO 2020/069429. In an embodiment, the conjugation density of Compound 101 in the modified alginate is determined by quantitative free amine analysis, e.g., as described in WO2020198695, wherein the determined conjugation density is 1.0% w/w to 3.0% w/w, 1.3% w/w to 2.8% w/w, 1.3% w/w to 2.6% w/w, 1.5% w/w to 2.4% w/w, 1.5% w/w to 2.2% w/w, or 1.7% w/w to 2.2% w/w.

A device, device preparation or device composition may be configured for implantation, or is implanted or disposed, into or onto any site or part of the body. In some embodiments, the implantable device or device preparation is configured for implantation into the peritoneal cavity (e.g., the lesser sac, also known as the omental bursa or bursalis omentum). A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, device preparation or device composition into the omentum (e.g., the lesser sac) are provided in M. Pellicciaro et al. (2017) CellR4 5(3):e2410.

### Device Manufacture

Genetically modified ARPE-19 cells for use in manufacturing a device described herein may be generated and cultured using methods known in the art. For example, stably-transfected ARPE-19 cells may be cultured in vitro substantially as described in WO2020198695.

Compounds of Formula (I) and alginates modified with such compounds may be obtained using procedures known in the art, e.g., substantially as those described in WO2020198695.

Alginate solutions for making afibrotic, two-compartment hydrogel capsules may be obtained using procedures known in the art, e.g., substantially as described in WO2020198695.

Two-compartment hydrogel capsules encapsulating in the inner compartment genetically modified mammalian cells and an afibrotic alginate in the outer layer (“shielded capsules”) may be generated using procedures known in the art, e.g., substantially as described in WO2020198696.

### Methods of Treatment

Described herein are methods for preventing or treating MPS-6 in a subject through administration or implantation of a pharmaceutical composition or genetically modified cells described herein. In an embodiment, the pharmaceutical composition comprises an ARSB fusion protein described herein and is formulated for intravenous or subcutaneous administration. In another embodiment, the pharmaceutical composition comprises a plurality of cells that are genetically modified to express the ARSB fusion protein. In an embodiment, the cells are encapsulated in hydrogel capsules described herein. In another embodiment, the cells are encapsulated in a macrodevice described herein. In some embodiments, the methods described herein directly or indirectly reduce or alleviate at least one symptom of MPS-6, or prevent or slow the onset of the disease. In an embodiment, the method comprises administering (e.g., implanting) an effective amount of a composition of two-compartment alginate hydrogel capsules which comprise in the inner compartment genetically modified RPE cells and a cell-binding polymer described herein and comprise a Compound of Formula (I), e.g., Compound 101, on the outer capsule surface and optionally within the outer compartment.

### EXAMPLES

In order that the disclosure described herein may be more fully understood, the following examples are set forth. The examples described in this application are offered to illustrate the ARSB fusion proteins and genetically modified cells provided herein and are not to be construed in any way as limiting their scope.

### Example 1: Effect of Fusing an Exemplary HSA-Binding Domain to a Human Mature ARSB on In Vitro ARSB Activity

DNA expression vectors were engineered to encode ARSB fusion enzymes containing an anti-HSA sdAb (R28) fused to the N-terminus or C-terminus of the mature human ARSB open reading frame via a (G4S)₃ linker unit. ARPE-19 cells were transfected with these expression vectors and polyclonal colonies stably expressing the encoded ARSB fusion enzyme were generated for each of the transfections.

The in vitro ARSB activity of the ARSB fusion proteins secreted from the two polyclonal colonies was assessed by seeding cells from each polyclonal colony at approximately 400,000 cells/well of a 6-well plate in 2 ml serum-rich medium (DMEM/F12/10% FBS+1 ug/ml puromycin) and were incubated in a temperature-controlled (TC) incubator (37 degrees C., 5% CO₂). Twenty to twenty-four hours post-seeding, the conditioned medium was collected and assessed for ARSB activity as described below.

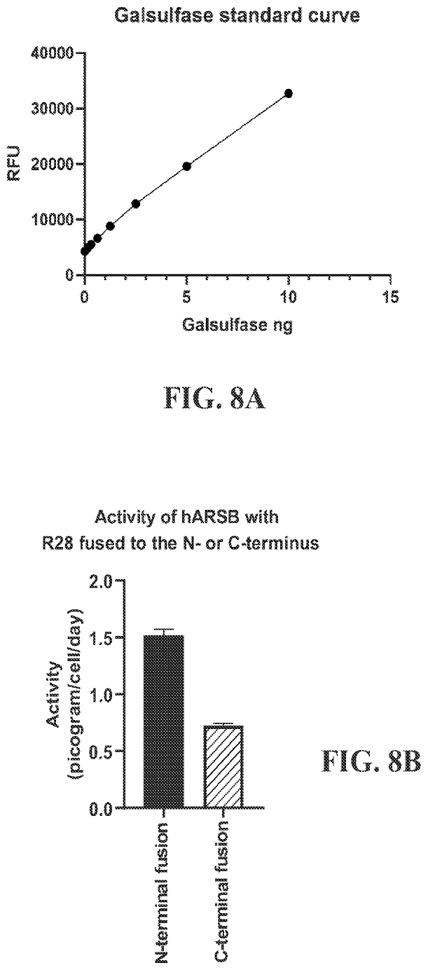

The conditioned medium was diluted 8-fold in Assay Buffer (50 mM sodium acetate, pH 5.6). Fifty microliters (ul) of the diluted conditioned medium were placed in a well of a 96-well black plate. Galsulfase as an exemplary recombinant human ARSB (rhARSB) was used as an activity standard. The rhARSB was serially diluted in Assay Matrix (composed of 1-part sterile serum-rich medium, 7-parts Assay Buffer) to generate a 7-point standard curve, with the top point set at 10 ng rhARSB in 50 ul Assay Matrix. The standard curve is shown in FIG. 8A.

Fifty microliters of 5 mM 4-Methylumbelliferyl Sulfate (4-MUS—Sigma Aldrich catalog #M7133-500MG) substrate were combined with 50 ul of diluted conditioned medium or with 50 ul rhARSB standard in the 96-well black plate. The reaction was incubated at ambient temperature in the dark for 1 hour. The reaction was then quenched in 100 ul glycine-carbonate buffer (12.8 grams glycine and 18 grams sodium carbonate dissolved in 200 ml molecular biology-grade water). The assay plate was scanned in a microplate reader for blue fluorescence at excitation and emission wavelengths of 365 nm and 445 nm (top read), respectively, in endpoint mode.

As shown in FIG. 8B, the ARSB activity produced by cells transfected with the vector encoding an R28-ARSB fusion protein was significantly higher than the ARSB activity produced by cells transfected with the vector encoding the ARSB-R28 fusion protein.

### Example 2: Exemplary Cell-Based ARSB Functional Assay: Quantifying Dermatan Sulfate Substrate Levels

MPS-6 patient fibroblasts (Coriell GM00538) are seeded at 250,000 cells/well of a 6-well plate in 2 ml serum-rich medium (EMEM/15% FBS) and incubated in a TC incubator (37 degrees C., 5% CO₂). Engineered ARPE-19 cells secreting an ARSB fusion protein are seeded at 400,000 cells/well of a 6-well plate in 2 ml non-selective, serum-rich medium (DMEM/F12/10% FBS—no puromycin) and then incubated in the TC incubator.

Twenty to twenty-four hours post-seeding, the conditioned medium are collected from the engineered cells and assessed for hARSB activity as described above. The conditioned medium containing an ARSB fusion protein described herein (“ARSB-fusion) is diluted to 500 ng ARSB-fusion per 1 ml sterile EMEM/15% FBS. Two milliliters of the 500 ng/ml ARSB-fusion solution is used to replace the conditioned medium of the MPS-6 patient fibroblasts seeded from the day before. The fibroblasts are incubated with the 2 ml solution of 500 ng/ml ARSB-fusion for 3 days in the TC incubator. After 3 days, the fibroblasts are rinsed in 1×PBS pH 7.4, collected via cell scraping, and lysed in 0.1% Triton-X 100 solution.

Ten microliters of cell lysates are combined with an equal volume of Chondroitinase Cocktail (0.8 ng/ul Chondroitinase B (R&D Systems #6974-GH) and 2 ng/ul Chondroitinase AC (R&D Systems)) in a buffer containing 100 mM Tris-Cl pH 7.5, 100 mM NaOAc, 10 mM CaCl₂, 20 mM MgCl₂/0.1% Triton-X 100). The reaction is incubated at in a thermocycler set to 20 degrees C. for 3 days. After 3 days, the entire reaction (20 ul) is combined with 180 ul of an aqueous acetonitrile solution [83.3 parts acetonitrile: 16.7 parts water] and clarified via centrifugation at 12,000 rpm for 10 minutes at 4 degrees C. One hundred microliters of the sample is analyzed for dermatan sulfate levels via LCMS.

### Example 3: Exemplary Cell-Based ARSB Functional Assay: Comparing Native and Half-Life Extended-hARSB Fusion Enzymes

In this example, the ability of native hARSB, half-life extended hARSB (HL-hARSB), and recombinant enzyme (rhARSB) to reduce chondroitin sulfate and dermatan sulfate were compared. Conditioned medium was prepared from cells expressing native hARSB, HL-hARSB, and rhARSB and tested on MPS VI patient-derived Fibroblasts according to the method of Example 2. As shown in FIG. 9, the HL-hARSB fusion protein shows similar biochemical characteristics to native and recombinant enzyme, successfully decreasing the amount of chondroitin sulfate/dermatan sulfate (CS/DS) compared to untreated controls.

### Example 4: Exemplary In Vivo Functional ARSB Functional Assay

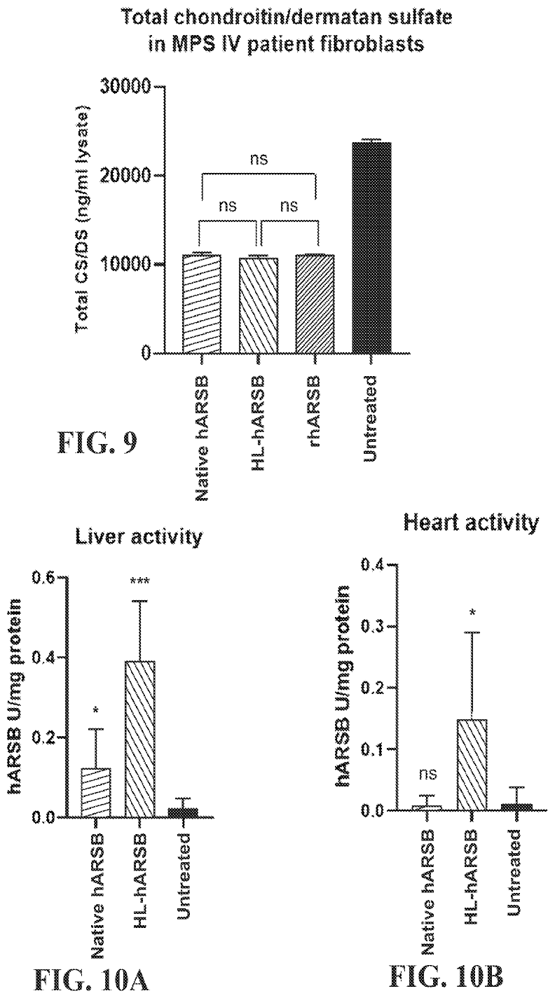

In this example, ARPE-19 cells engineered to express hARSB and HL-hARSB were encapsulated in afibrotic, two-compartment alginate hydrogel capsules as described in WO 2020/198695. The hydrogel capsules were implanted into MPS VI mice. After one week, organs were isolated from mice and activity was evaluated. As shown in FIGS. 10A-C, the total hARSB activity in liver, heart, and spleen is highest in mice treated with hydrogel capsules comprising cells engineered to express the HL-hARSB enzyme.

### Example 5: Exemplary In Vivo Functional ARSB Functional Assay—Evaluation of CS and DS Substrate Reduction in MPS VI Mouse Tissues

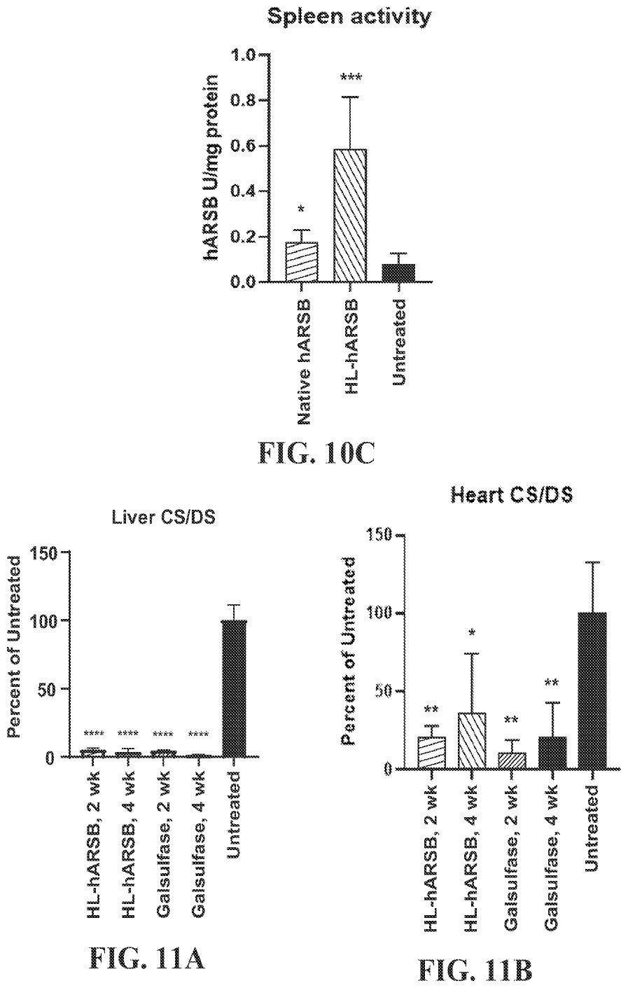

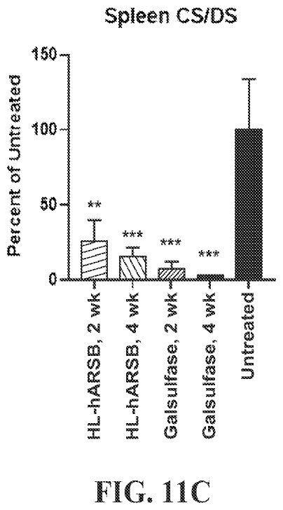

In this example, the ability of encapsulated HL-hARSB expressing cells to reduce CS/DS in mouse tissues was evaluated and compared to a positive control of recombinant protein (galsulfase). Hydrogel capsules were prepared as outlined in Example 4 and implanted into MPS VI mice. At two- and four-week time intervals, the mice were sacrificed and activity of CS/DS was measured in liver, heart, and spleen. As shown in FIGS. 11A-C, the total CS/DS activity in liver, heart, and spleen is comparable between the HL-hARSB and galsulfase groups in all tested conditions.

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A fusion protein which comprises an N-terminal to C-terminal structure defined by formula (F): AB-L-ARSB, wherein:
AB comprises a domain that binds to human serum albumin (HSA);
L, which may be present or absent, comprises a linker amino acid sequence; and
ARSB comprises an amino acid sequence for a mature mammalian ARSB protein.

2. The fusion protein of claim 1, wherein L is present.

3. The fusion protein of claim 1, wherein AB has a molecular weight of less than about any of 75 kDa, 50 kDa or 25 kDa.

4. The fusion protein of claim 1, wherein (i) AB comprises first, second and third amino acid sequences corresponding to the three complementarity determining regions CDR1, CDR2 and CDR3 of the heavy chain variable region of an anti-HSA antibody or (ii) AB comprises the CDR1, CDR2 and CDR3 sequences of the R11 sdAb, R28 sdAb, M75 sdAb or M79 sdAb set forth in Table 2A.

5. The fusion protein of claim 1, wherein AB comprises, consists essentially of, or consists of the amino acid sequence from a single domain antibody (sdAb), optionally wherein the amino acid sequence is selected from the group consisting of the R11, R28, M75 and M79 amino acid sequences disclosed in Table 2B, the Alb-1 and Alb-8 amino acid sequences described in Table II and Table III of WO 2006/22787 (e.g., SEQ ID NOs: 62-76).

6. The fusion protein of claim 1, wherein the AB CDR1 sequence is GRTFIAYA (SEQ ID NO:3) or a conservatively substituted variant thereof, the AB CDR2 sequence is ITNFAGGTT (SEQ ID NO:4) or a conservatively substituted variant thereof, and the AB CDR3 sequence is AADRSAQTMRQVRPVLPY (SEQ ID NO:5) or a conservatively substituted variant thereof.

7. The fusion protein of claim 1, wherein AB comprises, consists essentially of, or consists of the amino acid sequence from a sdAb.

8. The fusion protein of claim 7, wherein AB consists essentially of, or consists of, a parental or humanized sequence shown in Table 2B.

9. The fusion protein of claim 8, wherein AB consists essentially of, or consists of: QVQLVESGGGLVQAGGSLRLSCVASGRTFIAYAMGWFRQAPGKEREFVAAITNF AGGTTYYADSVKGRFTISRDNAKTTVYLQMNSLKPEDTALYYCAADRSAQTMR QVRPVLPYWGQGTQVTVSS (SEQ ID NO:6), or a conservatively substituted variant thereof.

10. The fusion protein of claim 8, wherein AB consists essentially of, or consists of: QVQLVESGGGLVQPGGSLRLSCAASGRTFIAYAMGWFRQAPGKEREFVAAITNF AGGTTYYADSVKGRFTISRDNAKTTVYLQMNSLRAEDTAVYYCAADRSAQTMR QVRPVLPYWGQGTLVTVSS (SEQ ID NO:7), or a conservatively substituted variant thereof.

11. The fusion protein of claim 1, wherein AB consists essentially of, or consists of, an amino acid sequence of the heavy chain variable region of an antibody that cross-competes with a sdAb consisting of SEQ ID NO:6 or SEQ ID NO:7 for binding to HSA.

12. The fusion protein of claim 1, wherein the fusion protein binds via AB to domain 1 (DI) or domain 2 (DII) of HSA and does not substantially inhibit binding of human FcRn to HSA.

13. The fusion protein of claim 1, wherein the fusion protein binds via the AB domain to HSA with a dissociation constant (K_{D}) affinity of less than about 0.1 nM to about 1,000 nM within a pH range of about 5.0 to about 7.4 as determined by surface plasmon resonance at 25° C.

14. The fusion protein of claim 13, wherein the fusion protein binds via the AB domain to HSA with a K_{D} of about 0.5 nM to about 500 nM, about 1 nM to about 250 nM, about 5 nM to about 50 nM, about 10 nM to about 25 nM, or about 0.5 nM to about 1 nM within a pH range of about 5.5 to about 7.4.

15. The fusion protein of claim 1, wherein the fusion protein binds via the AB domain to at least one mammalian serum albumin ortholog at 25° C. within a pH range of about 5.5 to about 7.4.

16. The fusion protein of claim 1, wherein fusion protein binds via AB to two or more mammalian serum albumins selected from the group consisting of mouse serum albumin, rat serum albumin, hamster serum albumin, rabbit serum albumin, guinea pig albumin, pig albumin, cat albumin, dog albumin, and a non-human primate serum albumin, optionally wherein the non-human primate serum albumin is cynomolgus serum albumin or rhesus monkey serum albumin.

17. The fusion protein of claim 16, wherein L is a linker peptide that is less than 50 amino acids in length, optionally wherein L is between about 10 and 30 amino acids, or between about 15 and 25 amino acids.

18. The fusion protein of claim 17, wherein L consists essentially of, or consists of, (GGGGS)n, wherein n is 3, 4 or 5.

19. The fusion protein of claim 18, wherein L consists essentially of, or consists of:
(SEQ ID NO: 9)
GGGGSGGGGSGGGGS.

20. The fusion protein of claim 1, wherein the mature mammalian protein comprises amino acids 37 to 533 of FIG. 1.

21. The fusion protein of claim 1, wherein the mature mammalian protein comprises amino acids 39 to 533 of FIG. 1.

22. The fusion protein of claim 1, which has an ARSB enzymatic activity that is within 80-120% of the corresponding activity of wild-type, mature human ARSB protein.

23. A polynucleotide which comprises a first nucleotide sequence that encodes the fusion protein of claim 1.

24. The polynucleotide of claim 23, wherein the first nucleotide sequence is operably linked to a nucleotide sequence encoding a secretory signal sequence for the fusion protein, optionally wherein the secretory signal sequence consists essentially of, or consists of, (i) MELGLSWVVLAALLQGVQA (SEQ ID NO:48) or (ii) one of the amino acid sequences set forth in Table 4.

25. The polynucleotide of claim 24, wherein the secretory signal sequence consists essentially of, or consists of MELGLSWVVLAALLQGVQA (SEQ ID NO:48).

26. The polynucleotide of claim 23, wherein the first nucleotide sequence is operably linked to a promoter sequence and a polyA signal sequence.

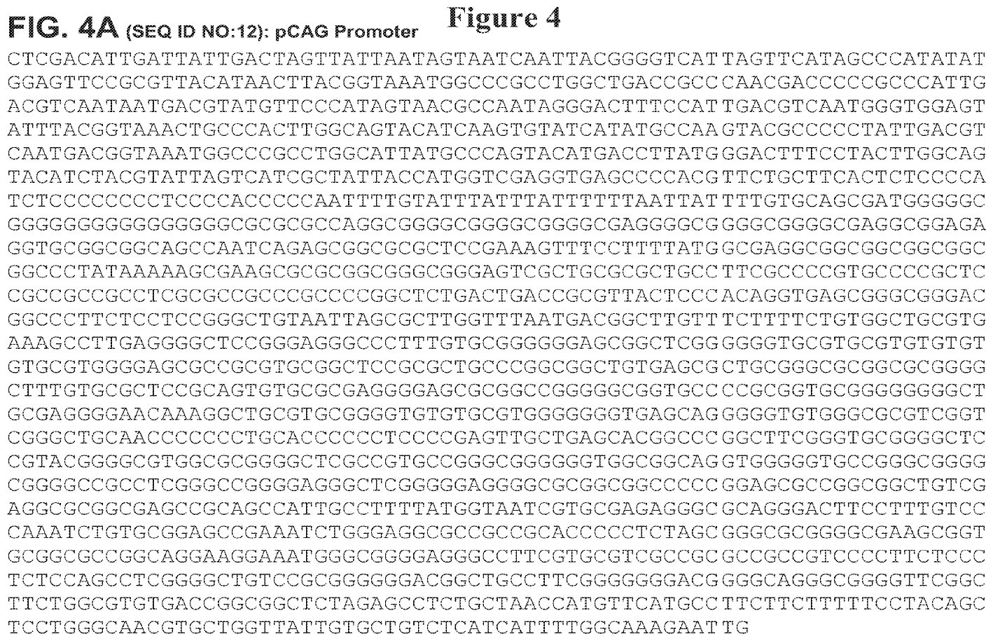

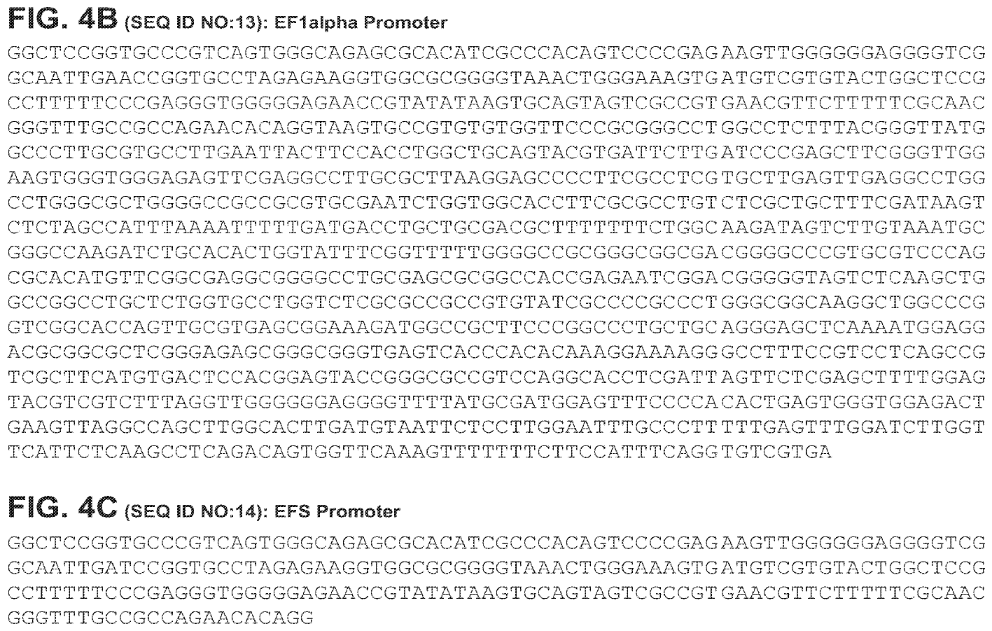

27. The polynucleotide of claim 26, wherein the promoter sequence is the pCAG promoter sequence shown in FIG. 4A (SEQ ID NO:12), the EF1α promoter sequence shown in FIG. 4B (SEQ ID NO:13) or the EFS promoter sequence shown in FIG. 4C (SEQ ID NO:14).

28. The polynucleotide of claim 26, wherein the promoter sequence is the EF1α promoter sequence shown in FIG. 4B (SEQ ID NO:13).

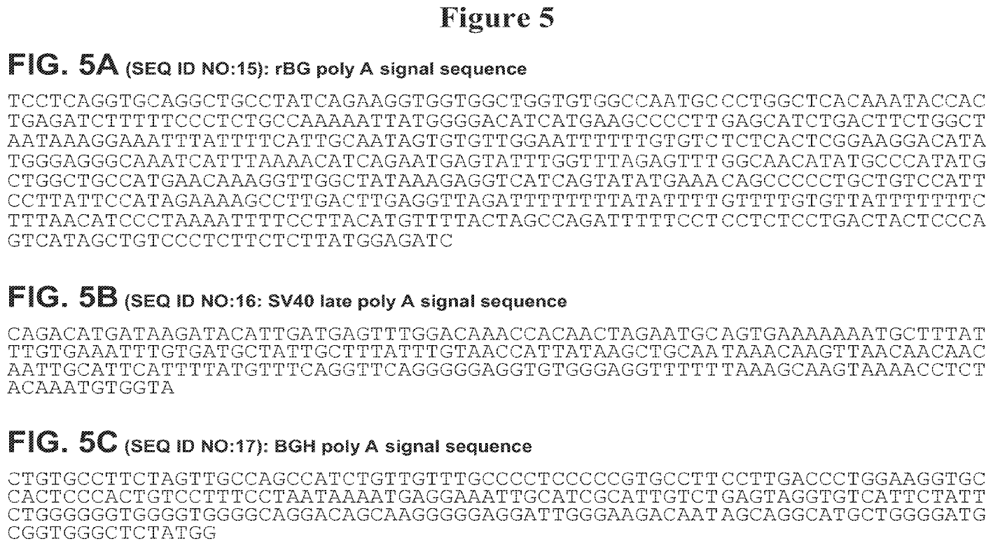

29. The polynucleotide of claim 26, wherein the polyA signal sequence is the rBG poly A signal sequence shown in FIG. 5A (SEQ ID NO:15), the SV40 late poly A signal sequence shown in FIG. 5B (SEQ ID NO:16) or the BGH poly A signal sequence shown in FIG. 5C (SEQ ID NO:17).

30. The polynucleotide of claim 29, wherein the polyA signal sequence is the rBG poly A signal sequence shown in FIG. 5A (SEQ ID NO:15).

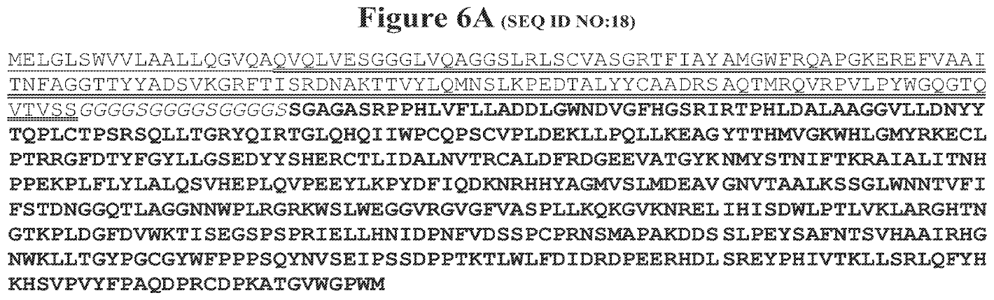

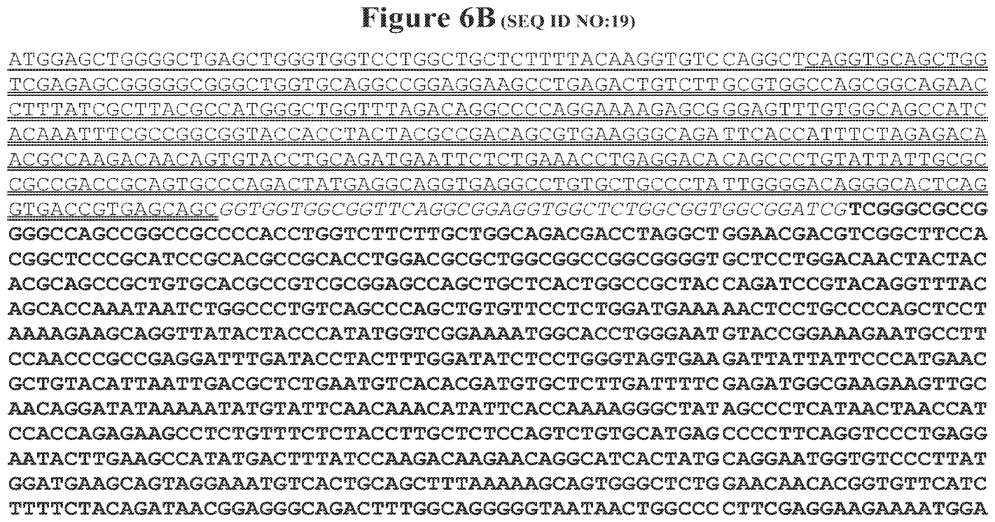

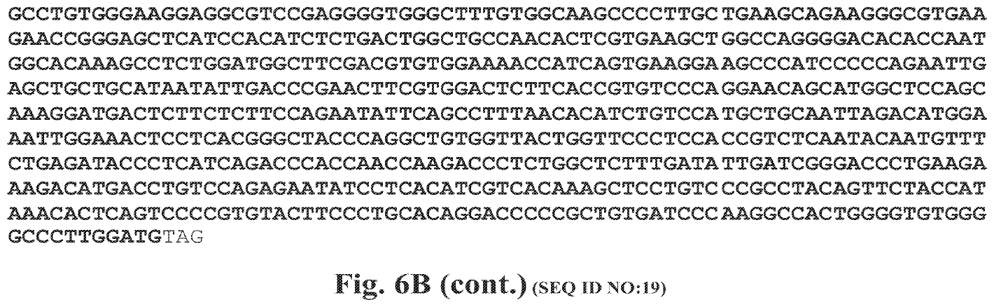

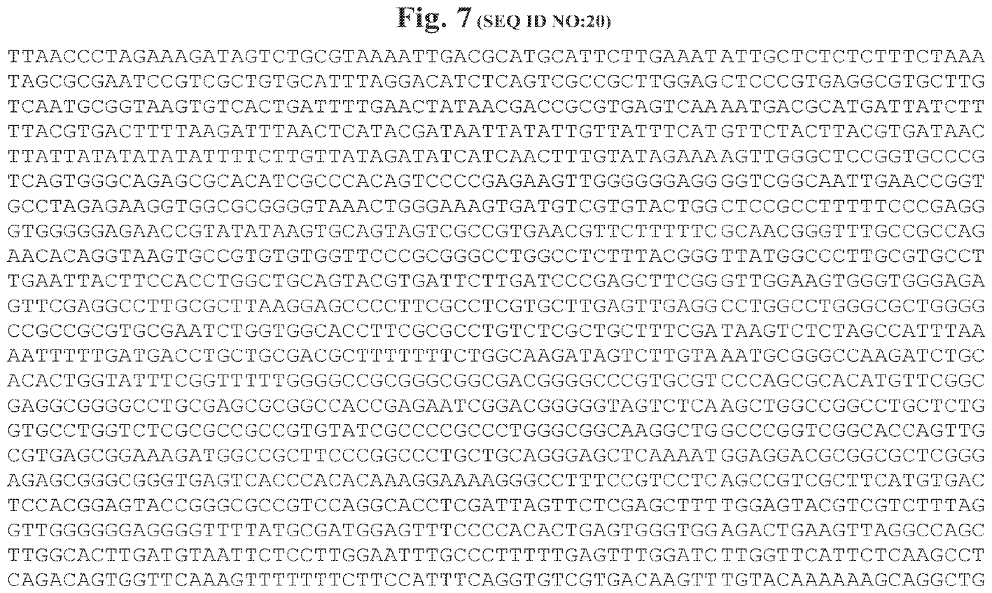

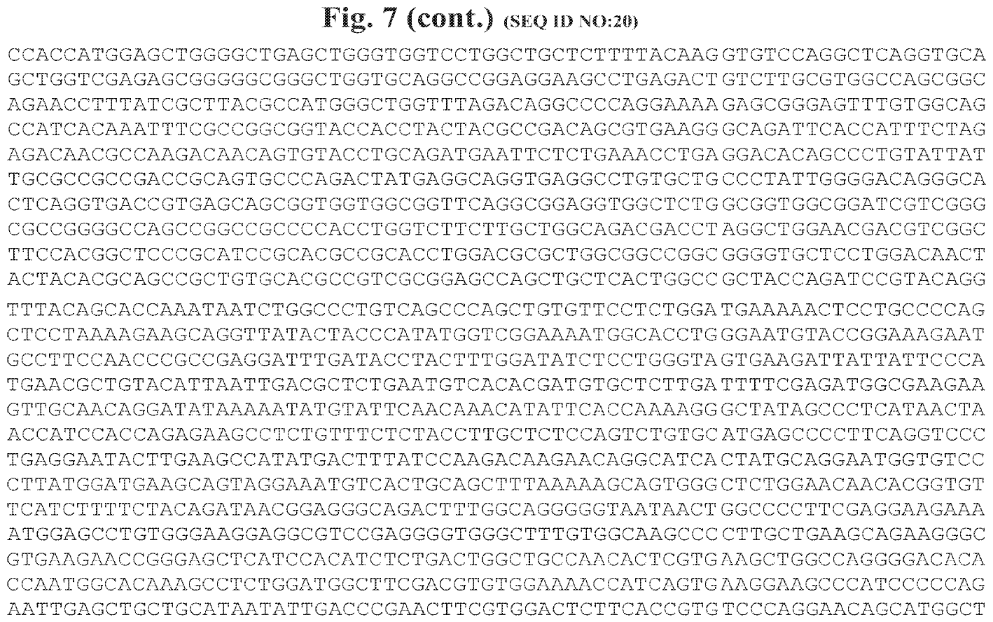

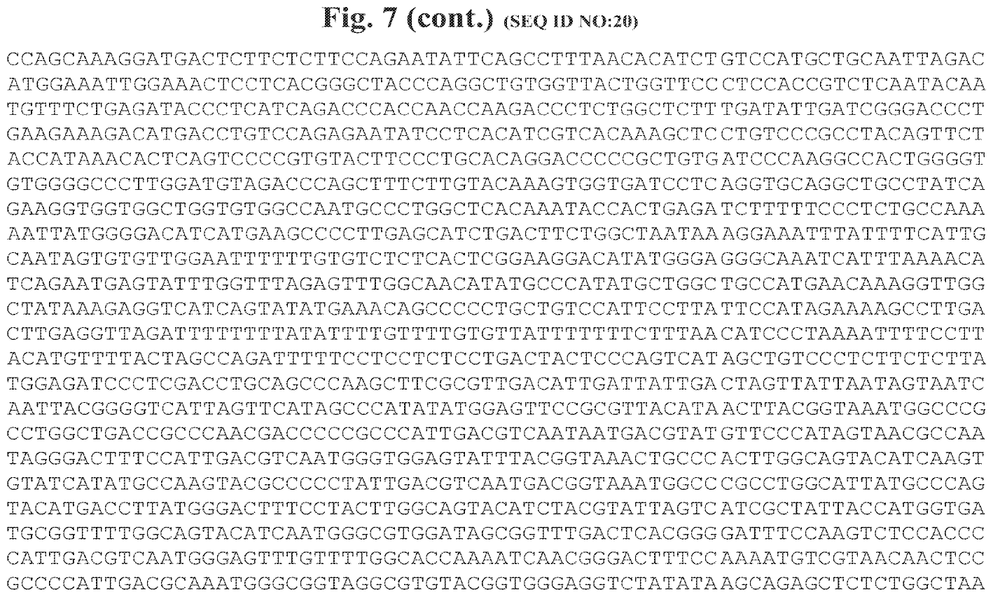

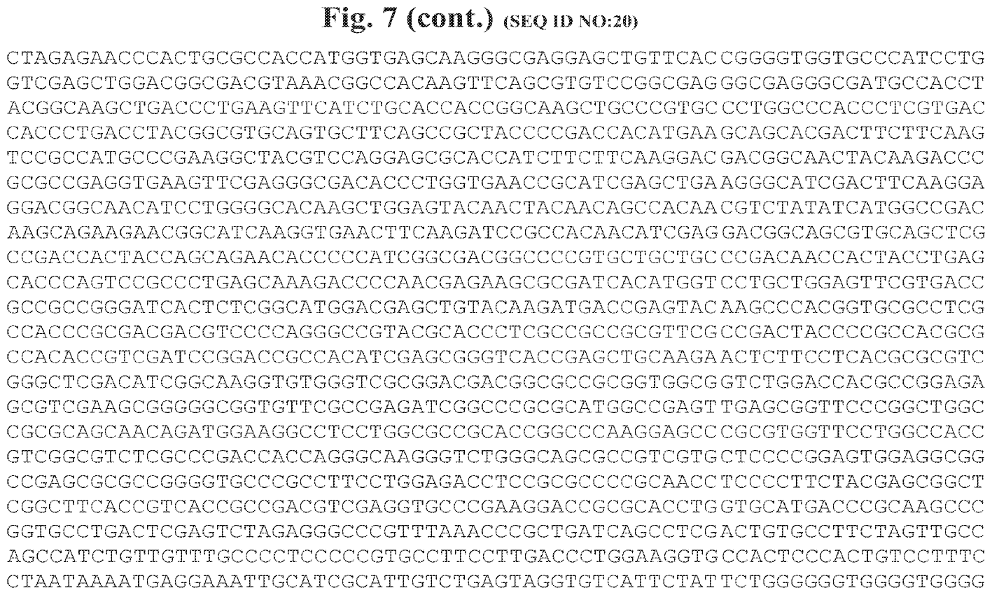

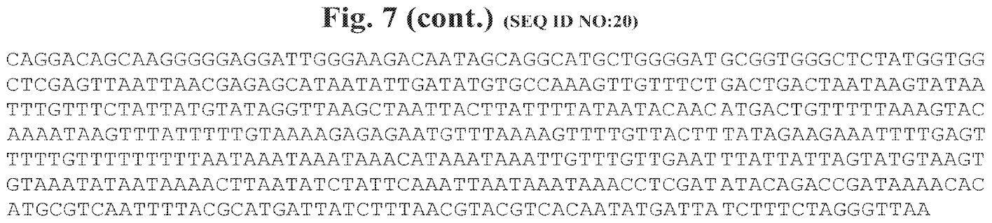

31. The polynucleotide of claim 30, which comprises the nucleotide sequence shown in FIG. 6B o(SEQ ID NO:19) or FIG. 7 (SEQ ID NO:20).

32. The polynucleotide of claim 23, which is one strand in an isolated double-stranded DNA molecule.

33. A genetically modified mammalian cell which is transiently or stably transfected with the polynucleotide of claim 23.

34. The genetically modified mammalian cell of claim 33, wherein the polynucleotide is inserted into at least one location in the genome of the mammalian cell.

35. The genetically modified mammalian cell of claim 33, wherein the cell is derived from a human cell.

36. The genetically modified mammalian cell of claim 35 which is derived from an RPE cell, optionally an ARPE-19 cell.

37. The genetically modified mammalian cell of claim 35, which is derived from an induced pluripotent stem cell (iPSC).

38. A composition comprising a plurality of genetically modified cells, wherein each cell in the plurality is a genetically modified cell as defined by claim 33.

39. The composition of claim 38, wherein the plurality of genetically modified cells is obtained from a culture of a monoclonal cell line.

40. An implantable device comprising at least one cell-containing compartment which comprises the genetically modified cell of any one of claim 33 or the composition of claim 38 and further comprises at least one means for mitigating the foreign body response (FBR) when the device is implanted into the subject.

41. The implantable device of claim 40, wherein the cell-containing compartment comprises a polymer composition, wherein the polymer composition comprises an alginate covalently modified with a peptide, wherein the peptide consists essentially of or consists of GRGDSP (SEQ ID NO:59), GGRGDSP (SEQ ID NO:60) or GGGRGDSP (SEQ ID NO:61).

42. The implantable device of claim 40, wherein the cell-containing compartment is surrounded by a barrier compartment comprising an alginate hydrogel and optionally a compound of Formula (I) disposed on the outer surface of the barrier compartment.

43. The implantable device of claim 41, wherein the polymer composition comprises an alginate covalently modified with a peptide, wherein the peptide consists essentially of or consists of GRGDSP (SEQ ID NO:59), and wherein the barrier compartment comprises an alginate chemically modified with 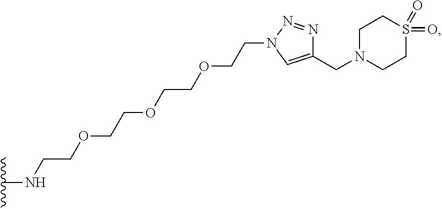 [SMILES: *NCCOCCOCCOCCn1cc(CN2CCS(=O)(=O)CC2)nn1]
or a pharmaceutically acceptable salt thereof.

44. The implantable device of claim 40, which is a spherical, two-compartment hydrogel capsule of about 0.75 mm to about 2 mm in diameter.

45. A preparation of devices, wherein each device in the preparation is a device of claim 40.

46. A hydrogel capsule comprising:
(a) an inner compartment which comprises a plurality of the genetically modified cell of any one of claims 33 to 37 encapsulated in a first polymer composition, wherein the first polymer composition comprises a hydrogel-forming polymer; and
(b) a barrier compartment surrounding the inner compartment and comprising a second polymer composition, wherein the second polymer composition comprises an alginate covalently modified with at least one compound of Formula (I) or a pharmaceutically acceptable salt thereof or a pharmaceutically acceptable salt of the compound.

47. The hydrogel capsule of claim 46, wherein the compound of Formula (I) is selected from a compound provided in the table below:

| Compound No. | Structure |
|---|---|
| 100 | 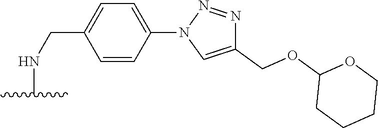 [SMILES: *NCc1ccc(-n2cc(COC3CCCCO3)nn2)cc1] |
| 101 | 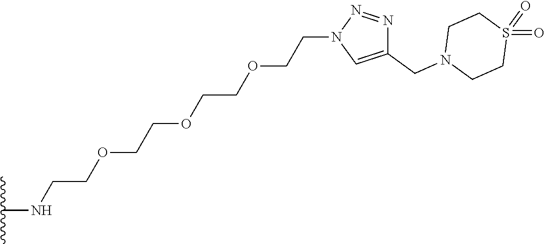 [SMILES: *NCCOCCOCCOCCn1cc(CN2CCS(=O)(=O)CC2)nn1] |
| 110 | 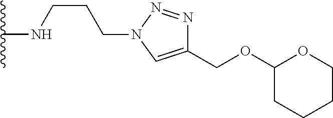 [SMILES: *NCCCn1cc(COC2CCCCO2)nn1] |
| 112 | 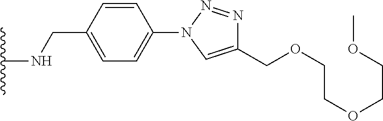 [SMILES: *NCc1ccc(-n2cc(COCCOCCOC)nn2)cc1] |
| 113 | 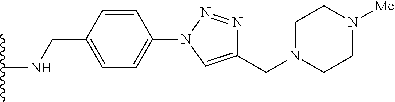 [SMILES: *NCc1ccc(-n2cc(CN3CCN(C)CC3)nn2)cc1] |
| 114 | 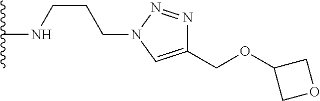 [SMILES: *NCCCn1cc(COC2COC2)nn1] |

48. The hydrogel capsule of claim 47, wherein the selected compound is

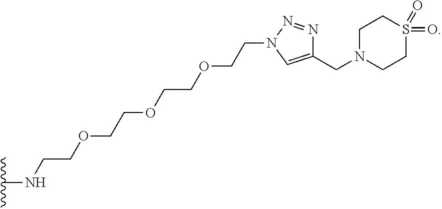 [SMILES: *NCCOCCOCCOCCn1cc(CN2CCS(=O)(=O)CC2)nn1]

49. The hydrogel capsule of claim 46, wherein the concentration of the genetically modified cell in the inner compartment is at least 40 million cells per ml of the first polymer composition.

50. A capsule composition comprising a plurality of the hydrogel capsule of claim 46, in a pharmaceutically acceptable carrier.

51. A pharmaceutical composition comprising the fusion protein of claim 1 and a pharmaceutically acceptable carrier.

52. A method of preventing or treating a subject with MPS-6, which comprises:
(i) administering to the subject the pharmaceutical composition of claim 51;
(ii) implanting the device or device preparation of claim 40; or
(iii) implanting the capsule composition of claim 50.

53. A method of treating a human subject diagnosed with MPS-6, comprising:
(a) providing the capsule composition of claim 50; and
(b) disposing the capsule composition in the body of the subject.

54. The method of claim 53, wherein the disposing step comprises placing the capsule composition into the intraperitoneal space of the subject.

55. The method of claim 53, wherein the disposing step comprises placing the capsule composition into the greater sac of the peritoneal cavity.
